(19) United States
(12) Patent Application Publication
(10) Pub. No.: US 2003/0083261 A1
(43) Pub. Date: May 1, 2003

(54) Class of 12mer peptides that inhibit the function of the mitotic check point protein Mad2

(76) Inventors: Hongtao Yu, Dallas, TX (US); Zhanyun Tang, Dallas, TX (US); Xuelian Luo, Dallas, TX (US); Jose Rizo-Rey, Dallas, TX (US)

Correspondence Address: Steven L. Highlander, Fulbright & Jaworski L.L.P., Suite 2400, 600 Congress Avenue, Austin, TX, 78701, US

(21) Appl. No.: 09/845,612

(22) Filed: Apr. 30, 2001

(51) Int. Cl. A61K 38/10; A61K 38/08

(52) U.S. Cl. 514/13; 514/14; 514/15; 514/16

(57) ABSTRACT

The present invention relates to methods of inhibiting Mad2 function by using a peptide that binds to Mad2, designated Mad2 binding peptides (MBPs). More particularly, the Mad2 binding peptides may be used to inhibit cancer cell proliferation. Yet further, Mad2 binding peptides may be used in combination with a second cancer therapy, for example taxol.

### BACKGROUND OF THE INVENTION

1. Field of the Invention

The present invention relates to fields of molecular biology and cancer. More particular the present invention relates to methods of inhibiting the function of a mitotic checkpoint protein, Mad2, using peptides that bind to Mad2, designated herein as Mad2-Binding Peptides (MBPs).

2. Description of Related Art

Tremendous progress has recently been made toward understanding the molecular mechanism of chromosome segregation. After all sister-chromatids have achieved bipolar attachment to the mitotic spindle, a ubiquitin ligase called the anaphase-promoting complex (APC), tags the securin protein with poly-ubiquitin chains (Nasmyth, 1999; King et al., 1995; Yu et al., 1998; Waizenegger et al., 2000). Degradation of the ubiquitinated securin by the 26S proteasome in turn activates the proteolytic activity of a protease called separase (Waizenegger et al., 2000; Uhlmann et al., 2000). Proteolytic cleavage of a cohesin subunit by separase destroys the cohesion between the sister-chromatids and triggers the onset of anaphase (Ciosk et al., 2000).

To ensure the high-fidelity transmission of the genetic material, the timing of sister-chromatid separation is closely monitored by the spindle assembly checkpoint, also known as the mitotic checkpoint (Rudner et al., 1996; Straight et al., 1997; Hardwick 1998). This checkpoint monitors for the proper attachment of chromosomes to the mitotic spindle and delays mitosis if this has not occurred correctly. The checkpoint senses the existence of kinetochores not yet occupied by microtubules (Gorbsky et al., 1993; Li et al., 1995; Nicklas et al., 1995). A single unattached kinetochore within a cell is sufficient to trigger this checkpoint (Nicklas, 1997) to prevent the initiation of anaphase. Anaphase-promoting complex (APC) is the target of the spindle checkpoint (Burke, 2000; Clarke et al., 2000). Inhibition of APC by the checkpoint leads to the stabilization of securin and prevents the premature separation of sister-chromatids until the proper attachment of all kinetochores to the spindle.

Several molecular components of this checkpoint pathway have been identified, including Mad1, Mad2, Mad3, Bub1, Bub2 and Bub3. These proteins were initially identified in S. cerevisiae; homologs of most of these proteins were then found in other organisms including vertebrates (Hoyt et al, 1991; Li et al. 1991; Roberts et al., 1994; Hardwick et al., 2000; Chen et al., 1996; Li et al., 1996; Taylor et al., 1997; Chen et al., 1998 Jin et al., 1998; Taylor et al., 1998). Interestingly, the vertebrate homologs of Mad1, Mad2, Bub1, and Bub3 localize to kinetochores during mitosis (Chen et al., 1996; Li et al., 1996; Taylor et al., 1997; Martinex-Exposito et al., 1999). In addition, a protein kinase called BubR1 that shares homology with both yeast Mad3 and Bub1, also resides on the kinetochores in mitosis (Taylor et al., 1998; Chan et al., 1998; Chan et al., 1999). Subsequent genetic and biochemical studies have shown that, with the exception of Bub2, all these molecules are involved in delaying the onset of anaphase in the presence of spindle damage and may partially account for the proper timing of chromosome segregation during normal mitosis (Taylor et al, 1997; Gardner et al., 2000).

Several lines of evidence suggest that defects of the spindle assembly checkpoint contributes to malignant transformation and tumorigenesis. First, the human Bub1 gene is mutated in human colorectal cancers, and Bub1 mutations may be responsible for the chromosomal instability and abnormal chromosome number (aneuploidy) observed in these tumors (Cahill et al., 1998). Second, mutations that inactivate murine homologs of Bub1 and Mad3 were found in the tumors of BRCA2 (Breast Cancer 2)-deficient mice. This implicates these mitotic checkpoint genes in the pathogenesis of inherited breast cancer (Lee et al., 1999). Third, Mad2 is expressed at lower levels in the breast cancer cell line T47D as compared to normal cells (Li et al, 1996). Finally, the viral oncoprotein Tax of human T cell leukemia virus type 1 (HTLV-1) binds to human Mad1 and compromises mitotic checkpoint function; this may be important for viral transformation (Jin et al., 1998). Taken together, these data indicate that the mitotic checkpoint is inactivated in many human cancers through various mechanisms, suggesting involvment in the frequent karyotypic abnormalities (aneuploidy) of tumor cells.

### SUMMARY OF THE INVENTION

Thus, in one aspect of the invention, there is provided a method of inhibiting Mad2 function comprising contacting a Mad2 protein with a peptide that binds Mad2. The peptide may be 9 to about 20 residues in length. In specific embodiments, the peptide is 12 residues in length.

The peptide comprises a core sequence represented by the formula X₁X₂X₃X₄X₅X₆X₇X₈X₉, wherein: X₁ can be any amino acid; X₂ and X₃ are hydrophobic residues; X₄ is a basic residue; X₅ is a hydrophobic residue; and at least one of X₆ to X₉ is P. Yet further, the peptide may comprise at least two P at X₆ to X₉. The peptide may also comprise at least one other P. Exemplary amino acid sequences of the peptide comprises, but is not limited to QWYKLX₆PP (SEQ ID NO:1), SWYSYPPPQRAV (SEQ ID NO:2), or DARIIKLPVPKP (SEQ ID NO:3).

In order to achieve effective inhibition of Mad2 protein function, the peptide may be present in a molar excess of Mad2, for example, but not limited to a 5-fold molar excess, a 10-fold molar excess, or a 100-fold molar excess.

The peptide may be delivered to a cell comprising Mad2. In order to be delivered to a cell, the peptide may be encapsulated in a liposome. Yet further, a nucleic acid encoding the peptide and a promoter may be delivered to a cell comprising Mad2. The promoter may be selected from the group consisting of CMV IE, RSV, and SV40 large T. The nucleic acid may further comprise a polyadenylation signal. The nucleic acid may be part of a replicable vector, for example a viral vector such as retroviral vector, adenoviral vector, adeno-associated viral vector, vaccinia viral vector, herpesviral vector and polyoma viral vector. The peptide may also be linked to a nuclear targeting molecule, such as a SV40 nuclear localization signal.

Mad2 may be located in a cancer cell. The cancer cell may be further contacted with a DNA damaging agent, for example radiation such as x-irradiation, γ-irradiation, uv-irradiation, and microwave irradiation. The cancer cell may also be treated with a microtubule inhibitor or an anti-mitotic agent. A specific microtubule inhibitor may be taxol.

In another embodiment, there is provided a method of inhibiting Mad2 function comprising contacting a Mad2 protein with a peptide-mimic that binds to Mad2.

In still another embodiment, there is provided a method of inhibiting cancer cell proliferation comprising contacting a Mad2 protein with a peptide or peptide-mimic that binds to Mad2. Examples of cancer cells include, but are not limited to a prostate cancer cell, a breast cancer cell, a lung cancer cell, a brain cancer cell, a liver cancer cell, a pancreatic cancer cell, a stomach cancer cell, a colon cancer cell, an ovarian cancer cell, a testicular cancer cell, a head & neck cancer cell, a throat cancer cell and an esophageal cancer cell. In specific embodiments, after the peptide contacts the Mad2 protein, the cancer cell stops growing, is killed, or is induced to undergo differentiation.

In yet another embodiment, there is provided a method of treating cancer in a subject comprising administering to cancer cells of the subject a peptide or peptide-mimic that binds to Mad2. The subject is a human. Yet further, a second cancer therapy may also be administered to the patient. The second cancer therapy may be DNA or microtubule damaging agents, for example, ionizing radiation or a chemotherapeutic agent. Specifically, the second cancer therapy is taxol.

In still a further embodiment, there is provided a method of screening for an anti-cancer agent comprising: providing a target polypeptide comprising at least the cdc20 binding domain of Mad2; contacting the target polypeptide with a candidate substance; determining the binding of the candidate substance to the target polypeptide; and in case of positive target polypeptide binding, screening for an anti-cancer effect. The candidate substance may be a peptide. The peptide may also be selected from a peptide library. Screening for an anti-cancer effect may comprise admixing the candidate substance with a cancer cell and measuring one or more characteristics of the cancer cell. For example, the characteristics may include cell growth, cell viability, cell shape or cell differentiation. Screening for an anti-cancer effect may also comprise contacting an expression vector encoding the peptide with a cancer cell and measuring one or more characteristics of the cancer cell. The target peptide may be expressed on the surface of a phage.

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIGS. 1A-B. Sequence alignment of Mad2-binding sequences. FIG. 1A shows the sequence alignment of the Mad2-binding sequences of Cdc20 from various organisms. (Hs, Homo Sapiens; DM, Drosophila; Sc, S. cerevisiae; Sp, S. pombe). FIG. 1B shows the identification of Mad2-binding peptides using phage display.

FIGS. 2A-B. Binding of MBP1 to Mad2 induces a dramatic conformational change. The structure of free Mad2 is shown on in FIG. 2A while a tentative 3D model of Mad2-MBP1 is shown in FIG. 2B.

FIGS. 3A-C. MBP1 blocks Mad2 function in vitro and in vivo. FIG. 3A shows that Mad2 inhibited the ubiquitination activity of APC using cyclin B1 as a substrate (compare lanes 2 & 3). Addition of 100 μM MBP1 blocked the ability of Mad2 to inhibit APC^{Cdc20} (lane 5) while a peptide containing the MBP1 sequence in reverse (MBP1-rev) had no effects (lane 4). FIG. 3B-C shows that overexpression of GFP-Mad2 in HeLa cells caused an arrest in mitosis whereas MBP1 counteracted the action of Mad2. HeLa cells were transfected with GFP-Mad2 plasmids, together with either a plasmid encoding MBP1 (FIG. 3B) or MBP1-rev (FIG. 3C). Cells in mitosis are round while the cells in interphase are flat.

### DESCRIPTION OF ILLUSTRATIVE EMBODIMENTS

The biochemical function of Mad2 is relatively well understood. Several lines of evidence have established that Mad2 binds directly to Cdc20, a WD40 repeat-containing protein that activates APC (Li et al., 1997; Fan et al., 1998; Kim et al., 1998; Hwang et al., 1998). Thus, Mad2 prevents the activation of APC and is the most downstream component of this checkpoint pathway. Among the other known checkpoint proteins, Bub1 and BubR1 are protein kinases and both interact with Bub3, another WD-40 repeat containing protein (Taylor et al., 1998). Mad1 is a coiled-coil protein and forms a tight complex with Mad2 throughout the cell cycle (Chen et al., 1998; Kim et al., 1998).

Experiments on mammalian cells have revealed two extraordinary features of the mitotic checkpoint. First, as a single unattached kinetochore can delay the onset of sister-chromatid separation, it must generate an inhibitory signal to block the activity of APC (Rieder et al., 1995). Moreover, this signal needs to be distributed throughout the cell to account for the inhibition of APC that is not associated with the unattached kinetochore (Shah et al., 2000).

Second, one of the traits of the unattached kinetochores that the checkpoint senses may be the lack of tension exerted by microtubules (Li et al., 1995). This notion is further strengthened by the recent finding that the kinesin-like motor, CENP-E, is an essential component of the mitotic checkpoint in mammalian cells and in Xenopus extracts (Abrieu et al., 2000 and Yao et al., 2000). CENP-E interacts directly with BubR1 in mitosis and this interaction is postulated to be a part of the force-sensing mechanism (Chan et al., 1999 and Yao et al, 2000).

The Mad2 protein is likely involved as the “wait anaphase” signal. First, Mad2 and Mad1 localize to unattached kinetochores (Li et al., 1996; Chen et al., 1998; and Chen et al., 1999). When the kinetochores are captured by microtubules, the concentrations of these proteins on the kinetochores drop sharply, suggesting that they play a direct role in generating the inhibitory signals. In contrast, the kinetochore localization of Bub1, BubR1, and Bub3 persists through anaphase (Martinez-Exposito et al., 1999 and Jablonski et al., 1998). Second, Mad2 interacts directly with Cdc20 and inhibits the activity of APC^{Cdc20} in vitro (Fang et al., 1998).

Mad2 turns over rapidly at the unattached kinetochores (Howell et al., 2000). Therefore, the unattached kinetochores may serve as catalytic sites for the generation of the active Mad2 species, which then diffuse away to inhibit APC. It is contemplated by the present invention that inhibition of Mad2 in cancer cells may result in improper chromosome segregation leading to apoptosis or cell death of the cancer cell. Using phage display, the inventors have identified peptides that bind to Mad2 or as referred to herein as Mad2-binding peptides (MBPs). These MBPs are the first known inhibitors of a mitotic checkpoint protein. It is envisioned that these MBPs may represent novel anti-cancer drugs. Specific aspects of the invention are described below.

1. MBP Peptides or Polypeptides The peptide sequences for Mad2-binding peptides (MBPs) comprise peptides that range from 9 to 20 residues in length. The preferred length in the present invention is 12 residues. The peptides may be generated synthetically or by recombinant techniques. The peptides may be purified according to known methods, such as precipitation (e.g., ammonium sulfate), HPLC, ion exchange chromatography, affinity chromatography (including immunoaffinity chromatography) or various size separations (sedimentation, gel electrophoresis, gel filtration).

A. Structural Features

The polypeptide sequences of the Mad2-binding peptides (MBPs) possess a consensus motif. The core of the motif consists of two hydrophobic residues, a basic residue, and a third hydrophobic residue. The hydrophobic residues tend to be aromatic amino acids. This core motif is generally followed by a proline-rich sequence. In the present invention the, core sequence is represented by the formula X₁X₂X₃X₄X₅X₆X₇X₈X₉. In specific embodiments, X₁ can be any amino acid; X₂ and X₃ are hydrophobic residues; X₄ is a basic residue; X₅ is a hydrophobic residue; and at least one of X₆ to X₉ is P. Particular peptides include QWYKLX₆PP (SEQ ID NO:1), SWYSYPPPQRAV (SEQ ID NO:2) and DARIIKLPVPKP (SEQ ID NO:3).

B. Amino Acid Design

One skilled in the art realizes that proteins or peptides can be engineered and utilized in place of the wild-type or native protein or peptide as long as the designed protein or peptide maintains a similar structure, charge, and function of the wild-type or native protein. The peptides of the present invention comprise a genus of peptides that have a core amino acid sequence represented by the formula contained herein. Thus, it is within the skill in the art to alter the peptides in this genus to enhance their function, e.g., binding ability to Mad2 or to enhance their stability in vivo or in vitro. Contained herein are some rules that may be considered in the design of peptides within this genus.

Substitutional variants typically contain the exchange of one amino acid for another at one or more sites within the protein, and may be designed to modulate one or more properties of the polypeptide, such as stability against proteolytic cleavage, without the loss of other functions or properties. Substitutions of this kind preferably are conservative, that is, one amino acid is replaced with one of similar shape and charge. Conservative substitutions are well known in the art and include, for example, the changes of: alanine to serine; arginine to lysine; asparagine to glutamine or histidine; aspartate to glutamate; cysteine to serine; glutamine to asparagine; glutamate to aspartate; glycine to proline; histidine to asparagine or glutamine; isoleucine to leucine or valine; leucine to valine or isoleucine; lysine to arginine; methionine to leucine or isoleucine; phenylalanine to tyrosine, leucine or methionine; serine to threonine; threonine to serine; tryptophan to tyrosine; tyrosine to tryptophan or phenylalanine; and valine to isoleucine or leucine.

The following is a discussion based upon changing of the amino acids of a peptide to create an equivalent, or even an improved, second-generation molecule. For example, certain amino acids may be substituted for other amino acids in a protein structure without appreciable loss of interactive binding capacity with structures such as, for example, antigen-binding regions of antibodies or binding sites on substrate molecules. Since it is the interactive capacity and nature of a peptide that defines that peptide's biological functional activity, certain amino acid substitutions can be made in a protein sequence, and its underlying DNA coding sequence, and nevertheless obtain a peptide with like properties. It is thus contemplated by the inventors that various changes may be made in the DNA sequences coding the peptide without appreciable loss of their biological utility or activity, as discussed below.

In making such changes, the hydropathic index of amino acids may be considered. The importance of the hydropathic amino acid index in conferring interactive biologic function on a protein is generally understood in the art (Kyte and Doolittle, 1982). It is accepted that the relative hydropathic character of the amino acid contributes to the secondary structure of the resultant peptide, which in turn defines the interaction of the peptide with other molecules.

Each amino acid has been assigned a hydropathic index on the basis of their hydrophobicity and charge characteristics (Kyte and Doolittle, 1982), these are: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5).

It is known in the art that certain amino acids may be substituted by other amino acids having a similar hydropathic index or score and still result in a peptide with similar biological activity, i.e., still obtain a biological functionally equivalent protein. In making such changes, the substitution of amino acids whose hydropathic indices are within ±2 is preferred, those which are within ±1 are particularly preferred, and those within ±0.5 are even more particularly preferred.

It is also understood in the art that the substitution of like amino acids can be made effectively on the basis of hydrophilicity. U.S. Pat. No. 4,554,101, incorporated herein by reference, states that the greatest local average hydrophilicity of a protein, as governed by the hydrophilicity of its adjacent amino acids, correlates with a biological property of the protein. As detailed in U.S. Pat. No. 4,554,101, the following hydrophilicity values have been assigned to amino acid residues: arginine (+3.0); lysine (+3.0); aspartate (+3.0±1); glutamate (+3.0±1); serine (+0.3); asparagine (+0.2); glutamine (+0.2); glycine (0); threonine (−0.4); proline (−0.5±1); alanine (−0.5); histidine *−0.5); cysteine (−1.0); methionine (−1.3); valine (−1.5); leucine (−1.8); isoleucine (−1.8); tyrosine (−2.3); phenylalanine (−2.5); tryptophan (−3.4).

It is understood that an amino acid can be substituted for another having a similar hydrophilicity value and still obtain a biologically equivalent and immunologically equivalent protein. In such changes, the substitution of amino acids whose hydrophilicity values are within ±2 is preferred, those that are within ±1 are particularly preferred, and those within ±0.5 are even more particularly preferred.

As outlined above, amino acid substitutions are generally based on the relative similarity of the amino acid side-chain substituents, for example, their hydrophobicity, hydrophilicity, charge, size, and the like. Exemplary substitutions that take various of the foregoing characteristics into consideration are well known to those of skill in the art and include: arginine and lysine; glutamate and aspartate; serine and threonine; glutamine and asparagine; and valine, leucine and isoleucine.

Another embodiment for the preparation of polypeptides according to the invention is the use of peptide mimetics. Mimetics are peptide containing molecules that mimic elements of protein secondary structure (Johnson et al., 1993). The underlying rationale behind the use of peptide mimetics is that the peptide backbone of proteins exists chiefly to orient amino acid side chains in such a way as to facilitate molecular interactions, such as those of antibody and antigen. A peptide mimetic is expected to permit molecular interactions similar to the natural molecule. These principles may be used, in conjunction with the principles outline above, to engineer second generation molecules having many of the natural properties of MBPs, but with altered and even improved characteristics.

C. Fusion Proteins

A specialized kind of insertional variant is the fusion protein. This molecule generally has all or a substantial portion of the native molecule, linked at the N- or C-terminus, to all or a portion of a second polypeptide. For example, fusions typically employ leader sequences from other species to permit the recombinant expression of a protein in a heterologous host. Another useful fusion includes the addition of a immunologically active domain, such as an antibody epitope, to facilitate purification of the fusion protein. Inclusion of a cleavage site at or near the fusion junction will facilitate removal of the extraneous polypeptide after purification. Other useful fusions include linking of functional domains, such as active sites from enzymes, glycosylation domains, cellular targeting signals or transmembrane regions. There also may be instances where a greater degree of intracellular specificity is desired. For example, with targeting nuclear proteins, RNA, DNA or cellular proteins or nucleic acids that are subsequently processed. Thus, one preferably uses localization sequences for such targets.

Localization sequences have been divided into routing signals, sorting signals, retention or salvage signals and membrane topology-stop transfer signals (Pugsley et al., 1989). For example, there are signals to target the endoplasmic reticulum (Munro, et al., 1987; Hangejorden et al., 1991), the nucleus (Lanford et al., 1986; Stanton et al., 1986; Harlow et al., 1985), the nucleolar region (Seomi et al., 1990; Kubota et al., 1989; and Siomi et al., 1988), the endosomal compartment (Bakke et al., 1990), mitochondria (Pugsley et al., 1989) and liposomes (Letourneur et al., 1992).

One preferred nuclear targeting sequence may be the SV40 nuclear localization signal.

D. Purification of Proteins

It may be desirable to purify MBPS, variants, peptide-mimics or analogs thereof. Protein purification techniques are well known to those of skill in the art. These techniques involve, at one level, the crude fractionation of the cellular milieu to polypeptide and non-polypeptide fractions. Having separated the polypeptide from other proteins, the polypeptide of interest may be further purified using chromatographic and electrophoretic techniques to achieve partial or complete purification (or purification to homogeneity). Analytical methods particularly suited to the preparation of a pure peptide are ion-exchange chromatography, exclusion chromatography; polyacrylamide gel electrophoresis; isoelectric focusing. A particularly efficient method of purifying peptides is fast protein liquid chromatography or even HPLC.

Certain aspects of the present invention concern the purification, and in particular embodiments, the substantial purification, of an encoded protein or peptide. The term “purified protein or peptide” as used herein, is intended to refer to a composition, isolatable from other components, wherein the protein or peptide is purified to any degree relative to its naturally-obtainable state. A purified protein or peptide therefore also refers to a protein or peptide, free from the environment in which it may naturally occur.

Generally, “purified” will refer to a protein or peptide composition that has been subjected to fractionation to remove various other components, and which composition substantially retains its expressed biological activity. Where the term “substantially purified” is used, this designation will refer to a composition in which the protein or peptide forms the major component of the composition, such as constituting about 50%, about 60%, about 70%, about 80%, about 90%, about 95% or more of the proteins in the composition.

Various methods for quantifying the degree of purification of the protein or peptide will be known to those of skill in the art in light of the present disclosure. These include, for example, determining the specific activity of an active fraction, or assessing the amount of polypeptides within a fraction by SDS/PAGE analysis. A preferred method for assessing the purity of a fraction is to calculate the specific activity of the fraction, to compare it to the specific activity of the initial extract, and to thus calculate the degree of purity, herein assessed by a “fold purification number.” The actual units used to represent the amount of activity will, of course, be dependent upon the particular assay technique chosen to follow the purification and whether or not the expressed protein or peptide exhibits a detectable activity.

Various techniques suitable for use in protein purification will be well known to those of skill in the art. These include, for example, precipitation with ammonium sulphate, PEG, antibodies and the like or by heat denaturation, followed by centrifugation; chromatography steps such as ion exchange, gel filtration, reverse phase, hydroxylapatite and affinity chromatography; isoelectric focusing; gel electrophoresis; and combinations of such and other techniques. As is generally known in the art, it is believed that the order of conducting the various purification steps may be changed, or that certain steps may be omitted, and still result in a suitable method for the preparation of a substantially purified protein or peptide.

There is no general requirement that the protein or peptide always be provided in their most purified state. Indeed, it is contemplated that less substantially purified products will have utility in certain embodiments. Partial purification may be accomplished by using fewer purification steps in combination, or by utilizing different forms of the same general purification scheme. For example, it is appreciated that a cation-exchange column chromatography performed utilizing an HPLC apparatus will generally result in a greater “-fold” purification than the same technique utilizing a low pressure chromatography system. Methods exhibiting a lower degree of relative purification may have advantages in total recovery of protein product, or in maintaining the activity of an expressed protein.

It is known that the migration of a polypeptide can vary, sometimes significantly, with different conditions of SDS/PAGE (Capaldi et al., 1977). It will therefore be appreciated that under differing electrophoresis conditions, the apparent molecular weights of purified or partially purified expression products may vary.

High Performance Liquid Chromatography (HPLC) is characterized by a very rapid separation with extraordinary resolution of peaks. This is achieved by the use of very fine particles and high pressure to maintain an adequate flow rate. Separation can be accomplished in a matter of minutes, or at most an hour. Moreover, only a very small volume of the sample is needed because the particles are so small and close-packed that the void volume is a very small fraction of the bed volume. Also, the concentration of the sample need not be very great because the bands are so narrow that there is very little dilution of the sample.

Gel chromatography, or molecular sieve chromatography, is a special type of partition chromatography that is based on molecular size. The theory behind gel chromatography is that the column, which is prepared with tiny particles of an inert substance that contain small pores, separates larger molecules from smaller molecules as they pass through or around the pores, depending on their size. As long as the material of which the particles are made does not adsorb the molecules, the sole factor determining rate of flow is the size. Hence, molecules are eluted from the column in decreasing size, so long as the shape is relatively constant. Gel chromatography is unsurpassed for separating molecules of different size because separation is independent of all other factors such as pH, ionic strength, temperature, etc. There also is virtually no adsorption, less zone spreading and the elution volume is related in a simple matter to molecular weight.

Affinity Chromatography is a chromatographic procedure that relies on the specific affinity between a substance to be isolated and a molecule that it can specifically bind to. This is a receptor-ligand type interaction. The column material is synthesized by covalently coupling one of the binding partners to an insoluble matrix. The column material is then able to specifically adsorb the substance from the solution. Elution occurs by changing the conditions to those in which binding will not occur (alter pH, ionic strength, temperature, etc.).

A particular type of affinity chromatography useful in the purification of carbohydrate containing compounds is lectin affinity chromatography. Lectins are a class of substances that bind to a variety of polysaccharides and glycoproteins. Lectins are usually coupled to agarose by cyanogen bromide. Conconavalin A coupled to Sepharose was the first material of this sort to be used and has been widely used in the isolation of polysaccharides and glycoproteins other lectins that have been include lentil lectin, wheat germ agglutinin which has been useful in the purification of N-acetyl glucosaminyl residues and Helix pomatia lectin. Lectins themselves are purified using affinity chromatography with carbohydrate ligands. Lactose has been used to purify lectins from castor bean and peanuts; maltose has been useful in extracting lectins from lentils and jack bean; N-acetyl-D galactosamine is used for purifying lectins from soybean; N-acetyl glucosaminyl binds to lectins from wheat germ; D-galactosamine has been used in obtaining lectins from clams and L-fucose will bind to lectins from lotus.

The matrix should be a substance that itself does not adsorb molecules to any significant extent and that has a broad range of chemical, physical and thermal stability. The ligand should be coupled in such a way as to not affect its binding properties. The ligand should also provide relatively tight binding. And it should be possible to elute the substance without destroying the sample or the ligand. One of the most common forms of affinity chromatography is immunoaffinity chromatography. The generation of antibodies that would be suitable for use in accord with the present invention is discussed below.

E. Peptide Synthesis

MBPs-related peptides may be generated synthetically for use in various embodiments of the present invention. Because of their relatively small size, the peptides of the invention can be synthesized in solution or on a solid support in accordance with conventional techniques. Various automatic synthesizers are commercially available and can be used in accordance with known protocols. See, for example, Stewart & Young, (1984); Tam et al, (1983); Merrifield, (1986); Barany & Merrifield (1979), each incorporated herein by reference. Short peptide sequences, or libraries of overlapping peptides, usually from about 6 up to about 35 to 50 amino acids, which correspond to the selected regions described herein, can be readily synthesized and then screened in screening assays designed to identify reactive peptides. Alternatively, recombinant DNA technology may be employed wherein a nucleotide sequence which encodes a peptide of the invention is inserted into an expression vector, transformed or transfected into an appropriate host cell and cultivated under conditions suitable for expression.

2. MBP Nucleic Acids

Important aspects of the present invention concern isolated DNA segments and recombinant vectors encoding MBPs and the creation and use of recombinant host cells through the application of DNA technology, that express a wild-type, polymorphic or mutant MBPs and biologically functional equivalents thereof.

The present invention concerns DNA segments, isolatable from mammalian cells, such as mouse, rat or human cells, that are free from total genomic DNA and that are capable of expressing a polypeptide or peptide. As used herein, the term “DNA segment” refers to a DNA molecule that has been isolated free of total genomic DNA of a particular species. Therefore, a DNA segment encoding MBPs refers to a DNA segment that contains wild-type, polymorphic or mutant MBPs coding sequences yet is isolated away from, or purified free from, total mammalian genomic DNA. Included within the term “DNA segment” are DNA segments and also recombinant vectors, including, for example, plasmids, cosmids, phage, viruses, and the like. One skilled in the art realizes that a polymorphic or mutant MBP is a biological functional equivalent of a MBP in that it binds to a mitotic checkpoint protein to inhibit its function resulting in improper segregation of chromosomes.

It will also be understood that amino acid and nucleic acid sequences may include additional residues, such as additional N- or C-terminal amino acids or 5′ or 3′ sequences, and yet still be essentially as set forth in one of the sequences disclosed herein, so long as the sequence meets the criteria set forth above, including the maintenance of biological protein, polypeptide or peptide activity where an amino acid sequence expression is concerned. The addition of terminal sequences particularly applies to nucleic acid sequences that may, for example, include various non-coding sequences flanking either of the 5′ or 3′ portions of the coding region or may include various internal sequences, i.e., introns, which are known to occur within genes.

3. Mutagenesis

In the design of peptides of the present invention, it may be necessary to utilize standard mutagenesis techniques. Mutagenesis may be used to screen for variants or analogs of MBP or Mad2-binding peptides. It is also envisioned that other peptides, variants or analogs thereof that bind to a different mitotic checkpoint protein may be isolated using standard mutagenesis techniques.

A. Chemical mutagenesis

Chemical mutagenesis offers certain advantages, such as the ability to find a full range of mutant alleles with degrees of phenotypic severity, and is facile and inexpensive to perform. The majority of chemical carcinogens produce mutations in DNA. Benzo[a]pyrene, N-acetoxy-2-acetyl aminofluorene and aflotoxin B1 cause GC to TA transversions in bacteria and mammalian cells. Benzo[a]pyrene also can produce base substitutions such as AT to TA. N-nitroso compounds produce GC to AT transitions. Alkylation of the O4 position of thymine induced by exposure to n-nitrosoureas results in TA to CG transitions.

B. In vitro Scanning Mutagenesis

Random mutagenesis also may be introduced using error prone PCR (Cadwell and Joyce, 1992). The rate of mutagenesis may be increased by performing PCR in multiple tubes with dilutions of templates.

One particularly useful mutagenesis technique is alanine scanning mutagenesis in which a number of residues are substituted individually with the amino acid alanine so that the effects of losing side-chain interactions can be determined, while minimizing the risk of large-scale perturbations in protein conformation (Cunningham et al., 1989).

In recent years, techniques for estimating the equilibrium constant for ligand binding using minuscule amounts of protein have been developed (Blackburn et al., 1991; U.S. Pat. Nos. 5,221,605 and 5,238,808). The ability to perform functional assays with small amounts of material can be exploited to develop highly efficient, in vitro methodologies for the saturation mutagenesis of antibodies. The inventors bypassed cloning steps by combining PCR mutagenesis with coupled in vitro transcription/translation for the high throughput generation of protein mutants. Here, the PCR products are used directly as the template for the in vitro transcription/translation of the mutant single chain antibodies. Because of the high efficiency with which all 19 amino acid substitutions can be generated and analyzed in this way, it is now possible to perform saturation mutagenesis on numerous residues of interest, a process that can be described as in vitro scanning saturation mutagenesis (Burks et al., 1997).

In vitro scanning saturation mutagenesis provides a rapid method for obtaining a large amount of structure-function information including: (i) identification of residues that modulate ligand binding specificity, (ii) a better understanding of ligand binding based on the identification of those amino acids that retain activity and those that abolish activity at a given location, (iii) an evaluation of the overall plasticity of an active site or protein subdomain, (iv) identification of amino acid substitutions that result in increased binding.

C. Random Mutagenesis by Fragmentation and Reassmbly

A method for generating libraries of displayed polypeptides is described in U.S. Pat. No. 5,380,721. The method comprises obtaining polynucleotide library members, pooling and fragmenting the polynucleotides, and reforming fragments therefrom, performing PCR amplification, thereby homologously recombining the fragments to form a shuffled pool of recombined polynucleotides.

D. Site-Directed Mutagenesis

Structure-guided site-specific mutagenesis represents a powerful tool for the dissection and engineering of protein-ligand interactions (Wells, 1996; Braisted et al, 1996). The technique provides for the preparation and testing of sequence variants by introducing one or more nucleotide sequence changes into a selected DNA.

Site-specific mutagenesis uses specific oligonucleotide sequences which encode the DNA sequence of the desired mutation, as well as a sufficient number of adjacent, unmodified nucleotides. In this way, a primer sequence is provided with sufficient size and complexity to form a stable duplex on both sides of the deletion junction being traversed. A primer of about 17 to 25 nucleotides in length is preferred, with about 5 to 10 residues on both sides of the junction of the sequence being altered.

The technique typically employs a bacteriophage vector that exists in both a single-stranded and double-stranded form. Vectors useful in site-directed mutagenesis include vectors such as the M13 phage. These phage vectors are commercially available and their use is generally well known to those skilled in the art. Double-stranded plasmids are also routinely employed in site-directed mutagenesis, which eliminates the step of transferring the gene of interest from a phage to a plasmid.

In general, one first obtains a single-stranded vector, or melts two strands of a double-stranded vector, which includes within its sequence a DNA sequence encoding the desired protein or genetic element. An oligonucleotide primer bearing the desired mutated sequence, synthetically prepared, is then annealed with the single-stranded DNA preparation, taking into account the degree of mismatch when selecting hybridization conditions. The hybridized product is subjected to DNA polymerizing enzymes such as E. coli polymerase I (Klenow fragment) in order to complete the synthesis of the mutation-bearing strand. Thus, a heteroduplex is formed, wherein one strand encodes the original non-mutated sequence, and the second strand bears the desired mutation. This heteroduplex vector is then used to transform appropriate host cells, such as E. coli cells, and clones are selected that include recombinant vectors bearing the mutated sequence arrangement.

Comprehensive information on the functional significance and information content of a given residue of protein can best be obtained by saturation mutagenesis in which all 19 amino acid substitutions are examined. The shortcoming of this approach is that the logistics of multiresidue saturation mutagenesis are daunting (Warren et al., 1996, Brown et al., 1996; Zeng et al., 1996; Burton and Barbas, 1994; Yelton et al, 1995; Jackson et al., 1995; Short et al., 1995; Wong et al, 1996; Hilton et al., 1996). Hundreds, and possibly even thousands, of site specific mutants must be studied. However, improved techniques make production and rapid screening of mutants much more straightforward. See also, U.S. Pat. Nos. 5,798,208 and 5,830,650, for a description of “walk-through” mutagenesis.

Other methods of site-directed mutagenesis are disclosed in U.S. Pat. Nos. 5,220,007; 5,284,760; 5,354,670; 5,366,878; 5,389,514; 5,635,377; and 5,789,166.

4. Screening Assays

The present invention also contemplates the screening of compounds, e.g., peptides, peptide-mimics, variants, analogs or small molecules, for various abilities to interact and/or affect expression or function of the mitotic checkpoint protein Mad2. Particularly preferred compounds will be those useful in inhibiting the action of Mad2. The compound may inhibit Mad2 by binding to the Mad2 protein. In the screening assays of the present invention, the candidate substance may first be screened for basic biochemical activity—e.g., binding to a target molecule (e.g., Mad2)—and then tested for its ability to inhibit function, at the cellular, tissue or whole animal level.

A. Modulators

The present invention provides methods of screening for modulators or inhibitors of Mad2 function. In an embodiment, the present invention is directed to a method of:

(a) providing a target polypeptide comprising at least the cdc20 binding domain of Mad2;

(b) contacting the target polypeptide with a candidate substance;

(c) determining the binding of the candidate substance to the target polypeptide; and

(d) in case of positive target polypeptide binding, screening for an anti-cancer effect.

In still yet other embodiments, one would look at the effect of a candidate substance as an anti-cancer agent. This can be done by examining cell growth, cell viability, cell shape or cell differentiation.

As used herein, the term “candidate substance” refers to any molecule that may potentially modulate Mad2 expression or function. The candidate substance may be a peptide, or a small molecule inhibitor, or even a nucleic acid molecule. It may prove to be the case that the most useful pharmacological compounds will be compounds that are structurally related to compounds which interact naturally with Mad2. Creating and examining the action of such molecules is known as “rational drug design,” and include making predictions relating to the structure of target molecules.

The goal of rational drug design is to produce structural analogs of biologically active polypeptides or target compounds. By creating such analogs, it is possible to fashion drugs which are more active or stable than the natural molecules, which have different susceptibility to alteration or which may affect the function of various other molecules. In one approach, one would generate a three-dimensional structure for a molecule like Mad2, and then design a molecule for its ability to interact with Mad2. Alternatively, one could design a partially functional fragment of Mad2 (binding, but no activity), thereby creating a competitive inhibitor. This could be accomplished by x-ray crystallography, computer modeling or by a combination of both approaches.

It also is possible to use antibodies to ascertain the structure of a target compound or inhibitor. In principle, this approach yields a pharmacore upon which subsequent drug design can be based. It is possible to bypass protein crystallography altogether by generating anti-idiotypic antibodies to a functional, pharmacologically active antibody. As a mirror image of a mirror image, the binding site of anti-idiotype would be expected to be an analog of the original antigen. The anti-idiotype could then be used to identify and isolate peptides from banks of chemically- or biologically-produced peptides. Selected peptides would then serve as the pharmacore. Anti-idiotypes may be generated using the methods described herein for producing antibodies, using an antibody as the antigen. On the other hand, one may simply acquire, from various commercial sources, small molecule libraries that are believed to meet the basic criteria for useful drugs in an effort to “brute force” the identification of useful compounds. Screening of such libraries, including combinatorially generated libraries (e.g., peptide libraries), is a rapid and efficient way to screen large number of related (and unrelated) compounds for activity. Combinatorial approaches also lend themselves to rapid evolution of potential drugs by the creation of second, third and fourth generation compounds modeled of active, but otherwise undesirable compounds. One may also screen a mutagenized population where the starting material is a MBP or an MBP consensus sequence.

Candidate compounds may include fragments or parts of naturally-occurring compounds or may be found as active combinations of known compounds which are otherwise inactive. It is proposed that compounds isolated from natural sources, such as animals, bacteria, fungi, plant sources, including leaves and bark, and marine samples may be assayed as candidates for the presence of potentially useful pharmaceutical agents. It will be understood that the pharmaceutical agents to be screened could also be derived or synthesized from chemical compositions or man-made compounds. Thus, it is understood that the candidate substance identified by the present invention may be a polypeptide, polynucleotide, small molecule inhibitor or any other compounds that may be designed through rational drug design starting from known inhibitors of hypertrophic response.

It will, of course, be understood that all the screening methods of the present invention are useful in themselves notwithstanding the fact that effective candidates may not be found. The invention provides methods for screening for such candidates, not solely methods of finding them.

B. In vitro Assays

A quick, inexpensive and easy assay to run is a binding assay. Binding of a molecule to a target may, in and of itself, be inhibitory, due to steric, allosteric or charge-charge interactions. This can be performed in solution or on a solid phase and can be utilized as a first round screen to rapidly eliminate certain compounds before moving into more sophisticated screening assays. In one embodiment of this kind, the screening of compounds that bind to a Mad2 molecule or fragment thereof is provided The target (e.g., Mad2) may be either free in solution, fixed to a support, expressed in or on the surface of a cell. Either the target or the compound may be labeled, thereby permitting determining of binding. Competitive binding assays can be performed in which one of the agents is labeled. Usually, the target will be the labeled species, decreasing the chance that the labeling will interfere with the binding moiety's function. One may measure the amount of free label versus bound label to determine binding or inhibition of binding.

A technique for high throughput screening of compounds is described in WO 84/03564. Large numbers of small peptide test compounds are synthesized on a solid substrate, such as plastic pins or some other surface. The peptide test compounds are reacted with, for example, Mad2 and washed. Bound polypeptide is detected by various methods.

Purified target can be coated directly onto plates for use in the aforementioned drug screening techniques. However, non-neutralizing antibodies to the polypeptide can be used to immobilize the polypeptide to a solid phase. Also, fusion proteins containing a reactive region may be used to link an active region to a solid phase.

C. In cyto Assays

Various cell lines that express Mad2 can be utilized for screening of candidate substances. Exemplary cell lines include, but are not limited to prostate cancer cells, breast cancer cells, lung cancer cells, brain cancer cells, liver cancer cells, pancreatic cancer cells, stomach cancer cells, colon cancer cells, ovarian cancer cells, testicular cancer cells, head & neck cancer cells, a throat cancer cell or esophageal cancer cells.

Cell lines containing wild-type, natural or mutated Mad2 may be engineered with indicators that can be used to study various functional attributes of candidate compounds. In such assays, the compound or MBP would be formulated appropriately, given its biochemical nature, and contacted with a target cell. Then, various biochemical, molecular or physiological properties may be measured. For example, but not limited to, measuring binding activity, mRNA levels, protein levels, nuclear stability, nuclear degradation, cell stability, cell differentiation, cell shape, enzymatic pathways, mitosis markers, chromosome degradation or apototsis markers.

In certain aspects of the present invention, cell lines may be engineered or transformed with two expression vectors, one expressing Mad2 and a second vector expressing an MBP.

These cell lines would be used to study the interaction of MBP with Mad2, inhibition of Mad2, viablity or proliferation of cells. It is also contemplated that cell lines that naturally contain Mad2 may be transfected with an expression vector containing an MBP. A lengthy discussion of expression vectors and methods of gene transfer therein is incorported into this section by reference.

Depending on the assay, culture may be required. As discussed above, the cell may then be examined by virtue of a number of different physiologic assays (growth, size, shape and differentiation). Alternatively, molecular analysis may be performed in which the function of Mad2 and the candidate substance and related pathways may be explored. This involves assays such as those for protein expression, protein function, substrate utilization, mRNA expression (including differential display of whole cell or polyA RNA) and others.

D. In vivo Assays

The present invention particularly contemplates the use of various animal models. For example, various cancer animal models may be used to determine if the inhibition of Mad2 effects the viability and proliferation of the cancer cells.

Treatment of these animals with test compounds will involve the administration of the compound, in an appropriate form, to the animal. Administration will be by any route the could be utilized for clinical or non-clinical purposes, including but not limited to oral, nasal, buccal, or even topical. Alternatively, administration may be by intratracheal instillation, bronchial instillation, intradermal, subcutaneous, intramuscular, intraperitoneal or intravenous injection. Specifically contemplated are systemic intravenous injection, regional administration via blood or lymph supply.

5. Engineering Expression Constructs

In certain embodiments, the present invention involves the manipulation of genetic material to produce expression constructs that encode Mad2 binding peptide (MBP). Such methods involve the generation of expression constructs containing, for example, a heterologous DNA encoding the peptide of interest and a means for its expression, replicating the vector in an appropriate helper cell, obtaining viral particles produced therefrom, and infecting cells with the recombinant virus particles.

A. Selectable Markers

In certain embodiments of the invention, the therapeutic expression constructs of the present invention contain nucleic acid constructs whose expression may be identified in vitro or in vivo by including a marker in the expression construct. Such markers would confer an identifiable change to the cell permitting easy identification of cells containing the expression construct. Usually the inclusion of a drug selection marker aids in cloning and in the selection of transformants. For example, genes that confer resistance to neomycin, puromycin, hygromycin, DHFR, GPT, zeocin and histidinol are useful selectable markers. Alternatively, enzymes such as herpes simplex virus thymidine kinase (tk) may be employed. Immunologic markers also can be employed. The selectable marker employed is not believed to be important, so long as it is capable of being expressed simultaneously with the nucleic acid encoding a gene product. Further examples of selectable markers are well known to one of skill in the art and include reporters such as EGFP, β-gal or chloramphenicol acetyltransferase (CAT).

B. Control Regions

Throughout this application, the term “expression construct” is meant to include any type of genetic construct containing a nucleic acid coding for the peptide of interest, such as a MBP.

The nucleic acid encoding the peptide or MBP is under transcriptional control of a promoter. A “promoter” refers to a DNA sequence recognized by the synthetic machinery of the cell, or introduced synthetic machinery, required to initiate the specific transcription of a gene. The phrase “under transcriptional control” means that the promoter is in the correct location and orientation in relation to the nucleic acid to control RNA polymerase initiation.

The term promoter will be used here to refer to a group of transcriptional control modules that are clustered around the initiation site for RNA polymerase II. Much of the thinking about how promoters are organized derives from analyses of several viral promoters, including those for the HSV thymidine kinase (tk) and SV40 early transcription units. These studies, augmented by more recent work, have shown that promoters are composed of discrete functional modules, each consisting of approximately 7-20 bp of DNA, and containing one or more recognition sites for transcriptional activator or repressor proteins.

At least one module in each promoter functions to position the start site for RNA synthesis. The best known example of this is the TATA box, but in some promoters lacking a TATA box, such as the promoter for the mammalian terminal deoxynucleotidyl transferase gene and the promoter for the SV40 late genes, a discrete element overlying the start site itself helps to fix the place of initiation.

Additional promoter elements regulate the frequency of transcriptional initiation. Typically, these are located in the region 30-110 bp upstream of the start site, although a number of promoters have recently been shown to contain functional elements downstream of the start site as well. The spacing between promoter elements frequently is flexible, so that promoter function is preserved when elements are inverted or moved relative to one another. In the tk promoter, the spacing between promoter elements can be increased to 50 bp apart before activity begins to decline. Depending on the promoter, it appears that individual elements can function either cooperatively or independently to activate transcription.

The particular promoter employed to control the expression of a nucleic acid sequence of interest is not believed to be important, so long as it is capable of directing the expression of the nucleic acid in the targeted cell. Thus, where a human cell is targeted, it is preferable to position the nucleic acid coding region adjacent to and under the control of a promoter that is capable of being expressed in a human cell. Generally speaking, such a promoter might include either a human or viral promoter.

In various embodiments, the human cytomegalovirus (CMV) immediate early gene promoter, the SV40 early promoter, the Rous sarcoma virus long terminal repeat, β-actin, rat insulin promoter and glyceraldehyde-3-phosphate dehydrogenase can be used to obtain high-level expression of the coding sequence of interest. The use of other viral or mammalian cellular or bacterial phage promoters which are well-known in the art to achieve expression of a coding sequence of interest is contemplated as well, provided that the levels of expression are sufficient for a given purpose. By employing a promoter with well-known properties, the level and pattern of expression of the protein of interest following transfection or transformation can be optimized.

Selection of a promoter that is regulated in response to specific physiologic or synthetic signals can permit inducible expression of the product. For example in the case where expression of a transgene, or transgenes when a multicistronic vector is utilized, is toxic to the cells in which the vector is produced in, it may be desirable to prohibit or reduce expression of one or more of the transgenes. Examples of transgenes that may be toxic to the producer cell line are pro-apoptotic and cytokine genes. Several inducible promoter systems are available for production of viral vectors where the transgene product may be toxic.

In some circumstances, it may be desirable to regulate expression of a transgene in a gene therapy vector. For example, different viral promoters with varying strengths of activity may be utilized depending on the level of expression desired. In mammalian cells, the CMV immediate early promoter if often used to provide strong transcriptional activation. Modified versions of the CMV promoter that are less potent have also been used when reduced levels of expression of the transgene are desired. When expression of a transgene in hematopoetic cells is desired, retroviral promoters such as the LTRs from MLV or MMTV are often used. Other viral promoters that may be used depending on the desired effect include SV40, RSV LTR, HIV-1 and HIV-2 LTR, adenovirus promoters such as from the E1A, E2A, or MLP region, AAV LTR, cauliflower mosaic virus, HSV-TK, and avian sarcoma virus.

Similarly tissue specific promoters may be used to effect transcription in specific tissues or cells so as to reduce potential toxicity or undesirable effects to non-targeted tissues. For example, promoters such as the PSA, probasin, prostatic acid phosphatase or prostate-specific glandular kallikrein (hK2) may be used to target gene expression in the prostate. Similarly, the following promoters may be used to target gene expression in other tissues (Table 1).

TABLE 1
Tissue specific promoters

| Tissue | Promoter |
|---|---|
| Pancreas | Insulin |
| | Elastin |
| | Amylase |
| | Pdr-1 pdx-1 |
| | Glucokinase |
| Liver | Albumin PEPCK |
| | HBV enhancer |
| | Alpha fetoprotein |
| | Apolipoprotein C |
| | Alpha-1 antitrypsin |
| | Vitellogenin, NF-AB |
| | Transthyretin |
| Skeletal muscle | Myosin H chain |
| | Muscle creatine kinase |
| | Dystrophin |
| | Calpain p94 |
| | Skeletal alpha-actin |
| | Fast troponin 1 |
| Skin | Keratin K6 |
| | Keratin K1 |
| Lung | CFTR |
| | Human cytokeratin 18 (K18) |
| | Pulmonary surfactant proteins A, B and C |
| | CC-10 |
| | P1 |
| Smooth muscle | Sm22 alpha |
| | SM-alpha-actin |
| Endothelium | Endothelin-1 |
| | E-selectin |
| | Von Willebrand factor |
| | TIE (Korhonen et al., 1995) |
| | KDR/flk-1 |
| Melanocytes | Tyrosinase |
| Adipose tissue | Lipoprotein lipase (Zechner et al., 1988) |
| | Adipsin (Spiegelman et al., 1989) |
| | Acetyl-CoA carboxylase (Pape and Kim, 1989) |
| | Glycerophosphate dehydrogenase (Dani et al., 1989) |
| | Adipocyte P2 (Hunt et al., 1986) |
| Blood | β-globin |

In certain indications, it may be desirable to activate transcription at specific times after administration of the gene therapy vector. This may be done with such promoters as those that are hormone or cytokine regulatable. For example in gene therapy applications where the indication is a gonadal tissue where specific steroids are produced or routed to, use of androgen or estrogen regulated promoters may be advantageous. Such promoters that are hormone regulatable include MMTV, MT-1, ecdysone and RuBisco. Other hormone regulated promoters such as those responsive to thyroid, pituitary and adrenal hormones are expected to be useful in the present invention. Cytokine and inflammatory protein responsive promoters that could be used include K and T Kininogen (Kageyama et al., 1987), c-fos, TNF-alpha, C-reactive protein (Arcone et al., 1988), haptoglobin (Oliviero et al., 1987), serum amyloid A2, C/EBP alpha, IL-1, IL-6 (Poli and Cortese, 1989), Complement C3 (Wilson et al., 1990), IL-8, alpha-1 acid glycoprotein (Prowse & Baumann, 1988), alpha-1 antitypsin, lipoprotein lipase (Zechner et al., 1988), angiotensinogen (Ron et al., 1991), fibrinogen, c-jun (inducible by phorbol esters, TNF-alpha, UV radiation, retinoic acid, and hydrogen peroxide), collagenase (induced by phorbol esters and retinoic acid), metallothionein (heavy metal and glucocorticoid inducible), Stromelysin (inducible by phorbol ester, interleukin-1 and EGF), alpha-2 macroglobulin and alpha-1 antichymotrypsin.

Promoters that could be used according to the present invention include Lac-regulatable, chemotherapy inducible (e.g. MDR), and heat (hyperthermia) inducible promoters, Radiation-inducible (e.g., EGR (Joki et al., 1995)), alpha-inhibin, RNA pol III tRNA met and other amino acid promoters, U1 snRNA (Bartlett et al., 1996), MC-1, PGK, and alpha-globin. Many other promoters that may be useful are listed in Walther & Stein (1996).

It is envisioned that any of the above promoters alone or in combination with another may be useful according to the present invention depending on the action desired. In addition, this list of promoters should not be construed to be exhaustive or limiting, those of skill in the art will know of other promoters that may be used in conjunction with the promoters and methods disclosed herein.

Enhancers are genetic elements that increase transcription from a promoter located at a distant position on the same molecule of DNA. Enhancers are organized much like promoters. That is, they are composed of many individual elements, each of which binds to one or more transcriptional proteins. The basic distinction between enhancers and promoters is operational. An enhancer region as a whole must be able to stimulate transcription at a distance; this need not be true of a promoter region or its component elements. On the other hand, a promoter must have one or more elements that direct initiation of RNA synthesis at a particular site and in a particular orientation, whereas enhancers lack these specificities. Promoters and enhancers are often overlapping and contiguous, often seeming to have a very similar modular organization.

Below is a list of promoters additional to the tissue specific promoters listed above, cellular promoters/enhancers and inducible promoters/enhancers that could be used in combination with the nucleic acid encoding a gene of interest in an expression construct (Table 2 and Table 3). Additionally, any promoter/enhancer combination (as per the Eukaryotic Promoter Data Base EPDB) could also be used to drive expression of the gene. Eukaryotic cells can support cytoplasmic transcription from certain bacterial promoters if the appropriate bacterial polymerase is provided, either as part of the delivery complex or as an additional genetic expression construct.

In preferred embodiments of the invention, the expression construct comprises a virus or engineered construct derived from a viral genome. The ability of certain viruses to enter cells via receptor-mediated endocytosis and to integrate into host cell genome and express viral genes stably and efficiently have made them attractive candidates for the transfer of foreign genes into mammalian cells (Ridgeway, 1988; Nicolas and Rubenstein, 1988; Baichwal and Sugden, 1986; Temin, 1986). The first viruses used as gene vectors were DNA viruses including the papovaviruses (simian virus 40, bovine papilloma virus, and polyoma) (Ridgeway, 1988; Baichwal and Sugden, 1986) and adenoviruses (Ridgeway, 1988; Baichwal and Sugden, 1986). These have a relatively low capacity for foreign DNA sequences and have a restricted host spectrum. Furthermore, their oncogenic potential and cytopathic effects in permissive cells raise safety concerns. They can accommodate only up to 8 kB of foreign genetic material but can be readily introduced in a variety of cell lines and laboratory animals (Nicolas and Rubenstein, 1988; Temin, 1986).

One will typically desire to include a polyadenylation signal to effect proper polyadenylation of the transcript. The nature of the polyadenylation signal is not believed to be crucial to the successful practice of the invention, and any such sequence may be employed such as human or bovine growth hormone and SV40 polyadenylation signals. Also contemplated as an element of the expression cassette is a terminator. These elements can serve to enhance message levels and to minimize read through from the cassette into other sequences.

TABLE 2
ENHANCER

Immunoglobulin Heavy Chain
Immunoglobulin Light Chain
T-Cell Receptor
HLA DQ α and DQ β
13-Interferon
Interleukin-2
Interleukin-2 Receptor
MHC Class II 5
MHC Class II HLA-DRα
β-Actin
Muscle Creatine Kinase
Prealbumin (Transthyretin)
Elastase I
Metallothionein
Collagenase
Albumin Gene
α-Fetoprotein
τ-Globin
β-Globin
e-fos
c-HA-ras
Insulin
Neural Cell Adhesion Molecule (NCAM)
α1 -Antitrypsin
H2B (TH2B) Histone
Mouse or Type I Collagen
Glucose-Regulated Proteins (GRP94 and GRP78)
Rat Growth Hormone
Human Serum Amyloid A (SAA)
Troponin I (TN I)
Platelet-Derived Growth Factor
Duchenne Muscular Dystrophy
SV40
Polyoma
Retroviruses
Papilloma Virus
Hepatitis B Virus
Human Immunodeficiency Virus
Cytomegalovirus
Gibbon Ape Leukemia Virus

TABLE 3

| Element | Inducer |
|---|---|
| MT II | Phorbol Ester (TPA) |
| | Heavy metals |
| MMTV (mouse mammary tumor | (Glucocorticoids |
| virus) | |
| β-interferon | Poly(rI)X |
| | Poly(rc) |
| Adenovirus 5 E2 | Ela |
| c-jun | Phorbol Ester (TPA), H₂O₂ |
| Collagenase | Phorbol Ester (TPA) |
| Stromelysin | Phorbol Ester (TPA), IL-1 |
| SV40 | Phorbol Ester (TPA) |
| Murine MX Gene | Interferon, Newcastle Disease Virus |
| GRP78 Gene | A23187 |
| β-2-Macroglobulin | IL-6 |
| Vimentin | Serum |
| MHC Class I Gene H-2kB | interferon |
| HSP70 | Ela, SV40 Large T Antigen |
| Proliferin | Phorbol Ester-TPA |
| Tumor Necrosis Factor | FMA |
| Thyroid Stimulating Hormone α | Thyroid Hormone |
| Gene | |
| Insulin E Box | Glucose |

6. Methods of Gene Transfer

In order to mediate the effect transgene expression in a cell, it will be necessary to transfer the therapeutic expression constructs of the present invention into a cell. Such transfer may employ viral or non-viral methods of gene transfer. This section provides a discussion of methods and compositions of gene transfer.

A. Viral Vector-Mediated Transfer

In certain embodiments, the nucleic acid sequence is incorporated into a viral particle to mediate gene transfer to a cell. Typically, the virus simply will be exposed to the appropriate host cell under physiologic conditions, permitting uptake of the virus. The present methods may be advantageously employed using a variety of viral vectors, as discussed below.

i) Adenovirus

Adenovirus is particularly suitable for use as a gene transfer vector because of its mid-sized DNA genome, ease of manipulation, high titer, wide target-cell range, and high infectivity. The roughly 36 kB viral genome is bounded by 100-200 base pair (bp) inverted terminal repeats (ITR), in which are contained cis-acting elements necessary for viral DNA replication and packaging. The early (E) and late (L) regions of the genome that contain different transcription units are divided by the onset of viral DNA replication.

The E1 region (E1A and E1B) encodes proteins responsible for the regulation of transcription of the viral genome and a few cellular genes. The expression of the E2 region (E2A and E2B) results in the synthesis of the proteins for viral DNA replication. These proteins are involved in DNA replication, late gene expression, and host cell shut off (Renan, 1990). The products of the late genes (L1, L2, L3, L4 and L5), including the majority of the viral capsid proteins, are expressed only after significant processing of a single primary transcript issued by the major late promoter (MLP). The MLP (located at 16.8 map units) is particularly efficient during the late phase of infection, and all the mRNAs issued from this promoter possess a 5′ tripartite leader (TL) sequence which makes them preferred mRNAs for translation.

In order for adenovirus to be optimized for gene therapy, it is necessary to maximize the carrying capacity so that large segments of DNA can be included. It also is very desirable to reduce the toxicity and immunologic reaction associated with certain adenoviral products. The two goals are, to an extent, coterminous in that elimination of adenoviral genes serves both ends. By practice of the present invention, it is possible achieve both these goals while retaining the ability to manipulate the therapeutic constructs with relative ease.

The large displacement of DNA is possible because the cis elements required for viral DNA replication all are localized in the inverted terminal repeats (ITR) (100-200 bp) at either end of the linear viral genome. Plasmids containing ITR's can replicate in the presence of a non-defective adenovirus (Hay et al., 1984). Therefore, inclusion of these elements in an adenoviral vector should permit replication.

In addition, the packaging signal for viral encapsidation is localized between 194-385 bp (0.5-1.1 map units) at the left end of the viral genome (Hearing et al., 1987). This signal mimics—the protein recognition site in bacteriophage λ DNA where a specific sequence close to the left end, but outside the cohesive end sequence, mediates the binding to proteins that are required for insertion of the DNA into the head structure. E1 substitution vectors of Ad have demonstrated that a 450 bp (0-1.25 map units) fragment at the left end of the viral genome could direct packaging in 293 cells (Levrero et al., 1991).

Previously, it has been shown that certain regions of the adenoviral genome can be incorporated into the genome of mammalian cells and the genes encoded thereby expressed. These cell lines are capable of supporting the replication of an adenoviral vector that is deficient in the adenoviral function encoded by the cell line. There also have been reports of complementation of replication deficient adenoviral vectors by “helping” vectors, e.g., wild-type virus or conditionally defective mutants.

Replication-deficient adenoviral vectors can be complemented, in trans, by helper virus. This observation alone does not permit isolation of the replication-deficient vectors, however, since the presence of helper virus, needed to provide replicative functions, would contaminate any preparation. Thus, an additional element was needed that would add specificity to the replication and/or packaging of the replication-deficient vector. That element, as provided for in the present invention, derives from the packaging function of adenovirus.

It has been shown that a packaging signal for adenovirus exists in the left end of the conventional adenovirus map (Tibbetts, 1977). Later studies showed that a mutant with a deletion in the E1A (194-358 bp) region of the genome grew poorly even in a cell line that complemented the early (E1A) function (Hearing and Shenk, 1983). When a compensating adenoviral DNA (0-353 bp) was recombined into the right end of the mutant, the virus was packaged normally. Further mutational analysis identified a short, repeated, position-dependent element in the left end of the Ad5 genome. One copy of the repeat was found to be sufficient for efficient packaging if present at either end of the genome, but not when moved towards the interior of the Ad5 DNA molecule (Hearing et al., 1987).

By using mutated versions of the packaging signal, it is possible to create helper viruses that are packaged with varying efficiencies. Typically, the mutations are point mutations or deletions. When helper viruses with low efficiency packaging are grown in helper cells, the virus is packaged, albeit at reduced rates compared to wild-type virus, thereby permitting propagation of the helper. When these helper viruses are grown in cells along with virus that contains wild-type packaging signals, however, the wild-type packaging signals are recognized preferentially over the mutated versions. Given a limiting amount of packaging factor, the virus containing the wild-type signals are packaged selectively when compared to the helpers. If the preference is great enough, stocks approaching homogeneity should be achieved.

ii) Retrovirus

The retroviruses are a group of single-stranded RNA viruses characterized by an ability to convert their RNA to double-stranded DNA in infected cells by a process of reverse-transcription (Coffin, 1990). The resulting DNA then stably integrates into cellular chromosomes as a provirus and directs synthesis of viral proteins. The integration results in the retention of the viral gene sequences in the recipient cell and its descendants. The retroviral genome contains three genes—gag, pol and env—that code for capsid proteins, polymerase enzyme, and envelope components, respectively. A sequence found upstream from the gag gene, termed Ψ, functions as a signal for packaging of the genome into virions. Two long terminal repeat (LTR) sequences are present at the 5′ and 3′ ends of the viral genome. These contain strong promoter and enhancer sequences and also are required for integration in the host cell genome (Coffin, 1990).

In order to construct a retroviral vector, a nucleic acid encoding a promoter is inserted into the viral genome in the place of certain viral sequences to produce a virus that is replication-defective. In order to produce virions, a packaging cell line containing the gag, pol and env genes but without the LTR and Ψ components is constructed (Mann et al., 1983). When a recombinant plasmid containing a human cDNA, together with the retroviral LTR and Ψ sequences is introduced into this cell line (by calcium phosphate precipitation for example), the Ψ sequence allows the RNA transcript of the recombinant plasmid to be packaged into viral particles, which are then secreted into the culture media (Nicolas and Rubenstein, 1988; Temin, 1986; Mann et al., 1983). The media containing the recombinant retroviruses is collected, optionally concentrated, and used for gene transfer. Retroviral vectors are able to infect a broad variety of cell types. However, integration and stable expression of many types of retroviruses require the division of host cells (Paskind et al., 1975).

An approach designed to allow specific targeting of retrovirus vectors recently was developed based on the chemical modification of a retrovirus by the chemical addition of galactose residues to the viral envelope. This modification could permit the specific infection of cells such as hepatocytes via asialoglycoprotein receptors, should this be desired.

A different approach to targeting of recombinant retroviruses was designed in which biotinylated antibodies against a retroviral envelope protein and against a specific cell receptor were used. The antibodies were coupled via the biotin components by using streptavidin (Roux et al., 1989). Using antibodies against major histocompatibility complex class I and class II antigens, the infection of a variety of human cells that bore those surface antigens was demonstrated with an ecotropic virus in vitro (Roux et al., 1989).

iii) Adeno-associated Virus

AAV utilizes a linear, single-stranded DNA of about 4700 base pairs. Inverted terminal repeats flank the genome. Two genes are present within the genome, giving rise to a number of distinct gene products. The first, the cap gene, produces three different virion proteins (VP), designated VP-1, VP-2 and VP-3. The second, the rep gene, encodes four non-structural proteins (NS). One or more of these rep gene products is responsible for transactivating AAV transcription.

The three promoters in AAV are designated by their location, in map units, in the genome. These are, from left to right, p5, p19 and p40. Transcription gives rise to six transcripts, two initiated at each of three promoters, with one of each pair being spliced. The splice site, derived from map units 42-46, is the same for each transcript. The four non-structural proteins apparently are derived from the longer of the transcripts, and three virion proteins all arise from the smallest transcript.

AAV is not associated with any pathologic state in humans. Interestingly, for efficient replication, AAV requires “helping” functions from viruses such as herpes simplex virus I and II, cytomegalovirus, pseudorabies virus and, of course, adenovirus. The best characterized of the helpers is adenovirus, and many “early” functions for this virus have been shown to assist with AAV replication. Low level expression of AAV rep proteins is believed to hold AAV structural expression in check, and helper virus infection is thought to remove this block.

The terminal repeats of the AAV vector can be obtained by restriction endonuclease digestion of AAV or a plasmid such as p201, which contains a modified AAV genome (Samulski et al., 1987), or by other methods known to the skilled artisan, including but not limited to chemical or enzymatic synthesis of the terminal repeats based upon the published sequence of AAV. The ordinarily skilled artisan can determine, by well-known methods such as deletion analysis, the minimum sequence or part of the AAV ITRs which is required to allow function, i.e., stable and site-specific integration. The ordinarily skilled artisan also can determine which minor modifications of the sequence can be tolerated while maintaining the ability of the terminal repeats to direct stable, site-specific integration.

AAV-based vectors have proven to be safe and effective vehicles for gene delivery in vitro, and these vectors are being developed and tested in pre-clinical and clinical stages for a wide range of applications in potential gene therapy, both ex vivo and in vivo (Carter and Flotte, 1996; Chatterjee et al., 1995; Ferrari et al., 1996; Fisher et al., 1996; Flotte et al., 1993; Goodman et al., 1994; Kaplitt et al., 1994; 1996, Kessler et al., 1996; Koeberl et al, 1997; Mizukami et al., 1996).

AAV-mediated efficient gene transfer and expression in the lung has led to clinical trials for the treatment of cystic fibrosis (Carter and Flotte, 1995; Flotte et al., 1993). Similarly, the prospects for treatment of muscular dystrophy by AAV-mediated gene delivery of the dystrophin gene to skeletal muscle, of Parkinson's disease by tyrosine hydroxylase gene delivery to the brain, of hemophilia B by Factor IX gene delivery to the liver, and potentially of myocardial infarction by vascular endothelial growth factor gene to the heart, appear promising since AAV-mediated transgene expression in these organs has recently been shown to be highly efficient (Fisher et al., 1996; Flotte et al., 1993; Kaplitt et al., 1994; 1996; Koeberl et al., 1997; McCown et al., 1996; Ping et al., 1996; Xiao et al., 1996).

iv) Other Viral Vectors

Other viral vectors may be employed as expression constructs in the present invention. Vectors derived from viruses such as vaccinia virus (Ridgeway, 1988; Baichwal and Sugden, 1986; Coupar et al., 1988) canary pox virus, and herpes viruses may be employed. These viruses offer several features for use in gene transfer into various mammalian cells.

B. Non-viral Transfer

Several non-viral methods for the transfer of expression constructs into cultured mammalian cells are contemplated by the present invention. These include calcium phosphate precipitation (Graham & Van Der Eb, 1973; Chen and Okayama, 1987; Rippe et al., 1990) DEAE-dextran (Gopal, 1985), electroporation (Tur-Kaspa et al., 1986; Potter et al., 1984), direct microinjection (Harland & Weintraub, 1985), DNA-loaded liposomes (Nicolau & Sene, 1982; Fraley et al., 1979), cell sonication (Fechheimer et al., 1987), gene bombardment using high velocity microprojectiles (Yang et al., 1990), and receptor-mediated transfection (Wu & Wu, 1987; Wu & Wu, 1988).

Once the construct has been delivered into the cell the nucleic acid encoding the therapeutic gene may be positioned and expressed at different sites. In certain embodiments, the nucleic acid encoding the therapeutic gene may be stably integrated into the genome of the cell. This integration may be in the cognate location and orientation via homologous recombination (gene replacement) or it may be integrated in a random, non-specific location (gene augmentation). In yet further embodiments, the nucleic acid may be stably maintained in the cell as a separate, episomal segment of DNA. Such nucleic acid segments or “episomes” encode sequences sufficient to permit maintenance and replication independent of or in synchronization with the host cell cycle. How the expression construct is delivered to a cell and where in the cell the nucleic acid remains is dependent on the type of expression construct employed.

In a particular embodiment of the invention, the expression construct may be entrapped in a liposome. Liposomes are vesicular structures characterized by a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers (Ghosh & Bachhawat, 1991). The addition of DNA to cationic liposomes causes a topological transition from liposomes to optically birefringent liquid-crystalline condensed globules (Radler et al., 1997). These DNA-lipid complexes are potential non-viral vectors for use in gene therapy.

Liposome-mediated nucleic acid delivery and expression of foreign DNA in vitro has been very successful. Using the β-lactamase gene, Wong et al., (1980) demonstrated the feasibility of liposome-mediated delivery and expression of foreign DNA in cultured chick embryo, HeLa, and hepatoma cells. Nicolau et al., (1987) accomplished successful liposome-mediated gene transfer in rats after intravenous injection. Also included are various commercial approaches involving “lipofection” technology.

In certain embodiments of the invention, the liposome may be complexed with a hemagglutinating virus (HVJ). This has been shown to facilitate fusion with the cell membrane and promote cell entry of liposome-encapsulated DNA (Kaneda et al., 1989). In other embodiments, the liposome may be complexed or employed in conjunction with nuclear nonhistone chromosomal proteins (HMG-1) (Kato et al., 1991). In yet further embodiments, the liposome may be complexed or employed in conjunction with both HVJ and HMG-1. In that such expression constructs have been successfully employed in transfer and expression of nucleic acid in vitro and in vivo, then they are applicable for the present invention.

Other vector delivery systems which can be employed to deliver a nucleic acid encoding a therapeutic gene into cells are receptor-mediated delivery vehicles. These take advantage of the selective uptake of macromolecules by receptor-mediated endocytosis in almost all eukaryotic cells. Because of the cell type-specific distribution of various receptors, the delivery can be highly specific (Wu & Wu, 1993).

Receptor-mediated gene targeting vehicles generally consist of two components: a cell receptor-specific ligand and a DNA-binding agent. Several ligands have been used for receptor-mediated gene transfer. The most extensively characterized ligands are asialoorosomucoid (ASOR) (Wu & Wu, 1987) and transferring (Wagner et al, 1990). Recently, a synthetic neoglycoprotein, which recognizes the same receptor as ASOR, has been used as a gene delivery vehicle (Ferkol et al., 1993; Perales et al., 1994) and epidermal growth factor (EGF) has also been used to deliver genes to squamous carcinoma cells (Myers, EPO 0273085). In other embodiments, the delivery vehicle may comprise a ligand and a liposome. For example, Nicolau et al., (1987) employed lactosyl-ceramide, a galactose-terminal asialganglioside, incorporated into liposomes and observed an increase in the uptake of the insulin gene by hepatocytes. Thus, it is feasible that a nucleic acid encoding a therapeutic gene also may be specifically delivered into a cell type such as prostate, epithelial or tumor cells, by any number of receptor-ligand systems with or without liposomes. For example, the human prostate-specific antigen (Watt et al., 1986) may be used as the receptor for mediated delivery of a nucleic acid in prostate tissue.

In another embodiment of the invention, the expression construct may simply consist of naked recombinant DNA or plasmids. Transfer of the construct may be performed by any of the methods mentioned above which physically or chemically permeabilize the cell membrane. This is applicable particularly for transfer in vitro, however, it may be applied for in vivo use as well. Dubensky et al. (1984) successfully injected polyomavirus DNA in the form of CaPO₄ precipitates into liver and spleen of adult and newborn mice demonstrating active viral replication and acute infection. Benvenisty and Neshif (1986) also demonstrated that direct intraperitoneal injection of CaPO₄ precipitated plasmids results in expression of the transfected genes. It is envisioned that DNA encoding a CAM also may be transferred in a similar manner in vivo and express CAM.

Another embodiment of the invention for transferring a naked DNA expression construct into cells may involve particle bombardment. This method depends on the ability to accelerate DNA coated microprojectiles to a high velocity allowing them to pierce cell membranes and enter cells without killing them (Klein et al., 1987). Several devices for accelerating small particles have been developed. One such device relies on a high voltage discharge to generate an electrical current, which in turn provides the motive force (Yang et al., 1990). The microprojectiles used have consisted of biologically inert substances such as tungsten or gold beads.

7. Methods for Treating Cancer

The present invention also contemplates inhibiting Mad2 function. Inhibition of Mad2 funtion would result in improper chromosome segregation leading to cell death. Thus, it is contemplated that the introduction of the MBPs, peptide-mimics or analogs thereof into cancer cells would promote cell death of the cancer cells. It is also envisioned that MBPs or analogs thereof would interfere with the stablilty of the cancer cell leaving the cancer cells susceptible to traditional cancer treatments.

A. Genetic Based Therapies

Specifically, the present inventors intend to provide, to a cell, an expression construct capable of providing MBPs to that cell. The lengthy discussion of expression vectors and the genetic elements employed therein is incorporated into this section by reference. Particularly preferred expression vectors are viral vectors such as adenovirus, adeno-associated virus, herpesvirus, vaccinia virus and retrovirus. Also preferred is liposomally-encapsulated expression vector.

Those of skill in the art are well aware of how to apply gene delivery to in vivo and ex vivo situations. For viral vectors, one generally will prepare a viral vector stock. Depending on the kind of virus and the titer attainable, one will deliver 1×10⁴, 1×10⁵, 1×10⁶, 1×10⁷, 1×10⁸, 1×10⁹, 1×10¹⁰, 1×10¹¹ or 1×10¹² infectious particles to the patient. Similar FIGS. may be extrapolated for liposomal or other non-viral formulations by comparing relative uptake efficiencies. Formulation as a pharmaceutically acceptable composition is discussed below.

B. Protein Therapy

Another therapy approach is the provision, to a subject, of MBPs, synthetic peptides, mimetics or analogs thereof. The protein may be produced by recombinant expression means. Formulations would be selected based on the route of administration and purpose including, but not limited to, liposomal formulations and classic pharmaceutical preparations.

C. Combined Therapy

In order to increase the effectiveness of the MBPs or peptide-mimic or analog thereof, it may be desirable to combine these compositions with an agent effective in the treatment of hyperproliferative disease, such as, for example, an anti-cancer agent. An “anti-cancer” agent is capable of negatively affecting cancer in a subject, for example, by killing one or more cancer cells, inducing apoptosis in one or more cancer cells, reducing the growth rate of one or more cancer cells, reducing the incidence or number of metastases, reducing a tumor's size, inhibiting a tumor's growth, reducing the blood supply to a tumor or one or more cancer cells, promoting an immune response against one or more cancer cells or a tumor, preventing or inhibiting the progression of a cancer, or increasing the lifespan of a subject with a cancer. Anti-cancer agents include, for example, chemotherapy agents (chemotherapy), radiotherapy agents (radiotherapy), a surgical procedure (surgery), immune therapy agents (immunotherapy), genetic therapy agents (gene therapy), hormonal therapy, other biological agents (biotherapy) and/or alternative therapies.

More generally, such an agent would be provided in a combined amount with an effective amount of either a MBP, a peptide-mimic or an analog to kill or inhibit proliferation of a cancer cell. This process may involve contacting the cell(s) with an agent(s) and the MBP or peptide-mimic or analog at the same time or within a period of time wherein separate administration of the MBP or peptide-mimic or analog and an agent to a cell, tissue or organism produces a desired therapeutic benefit. This may be achieved by contacting the cell, tissue or organism with a single composition or pharmacological formulation that includes both a MBP or peptide-mimic or analog and one or more agents, or by contacting the cell with two or more distinct compositions or formulations, wherein one composition includes a MBP or peptide-mimic or analog and the other includes one or more agents.

The terms “contacted” and “exposed,” when applied to a cell, tissue or organism, are used herein to describe the process by which a therapeutic construct of MBP or peptide-mimic or analog and/or another agent, such as for example a chemotherapeutic or radiotherapeutic agent, are delivered to a target cell, tissue or organism or are placed in direct juxtaposition with the target cell, tissue or organism. To achieve cell killing or stasis, the MBP or peptide-mimic or analog and/or additional agent(s) are delivered to one or more cells in a combined amount effective to kill the cell(s) or prevent them from dividing.

The MBP or peptide-mimic or analog may precede, be co-current with and/or follow the other agent(s) by intervals ranging from minutes to weeks. In embodiments where the MBP or peptide-mimic or analog, and other agent(s) are applied separately to a cell, tissue or organism, one would generally ensure that a significant period of time did not expire between the time of each delivery, such that the MBP or peptide-mimic or analog and agent(s) would still be able to exert an advantageously combined effect on the cell, tissue or organism. For example, in such instances, it is contemplated that one may contact the cell, tissue or organism with two, three, four or more modalities substantially simultaneously (i.e. within less than about a minute) as the MBP or peptide-mimic or analog. In other aspects, one or more agents may be administered within of from substantially simultaneously, about 1 minute, about 5 minutes, about 10 minutes, about 20 minutes about 30 minutes, about 45 minutes, about 60 minutes, about 2 hours, about 3 hours, about 4 hours, about 5 hours, about 6 hours, about 7 hours about 8 hours, about 9 hours, about 10 hours, about 11 hours, about 12 hours, about 13 hours, about 14 hours, about 15 hours, about 16 hours, about 17 hours, about 18 hours, about 19 hours, about 20 hours, about 21 hours, about 22 hours, about 22 hours, about 23 hours, about 24 hours, about 25 hours, about 26 hours, about 27 hours, about 28 hours, about 29 hours, about 30 hours, about 31 hours, about 32 hours, about 33 hours, about 34 hours, about 35 hours, about 36 hours, about 37 hours, about 38 hours, about 39 hours, about 40 hours, about 41 hours, about 42 hours, about 43 hours, about 44 hours, about 45 hours, about 46 hours, about 47 hours, about 48 hours, about 1 day, about 2 days, about 3 days, about 4 days, about 5 days, about 6 days, about 7 days, about 8 days, about 9 days, about 10 days, about 11 days, about 12 days, about 13 days, about 14 days, about 15 days, about 16 days, about 17 days, about 18 days, about 19 days, about 20 days, about 21 days, about 1, about 2, about 3, about 4, about 5, about 6, about 7 or about 8 weeks or more, and any range derivable therein, prior to and/or after administering the MBP or peptide-mimic or analog.

Various combination regimens of the MBP or peptide-mimic or analog and one or more agents may be employed. Non-limiting examples of such combinations are shown below, wherein a composition MBP or peptide-mimic or analog is “A” and an agent is “B”:

| | | | | | |
|---|---|---|---|---|---|
| A/B/A | B/A/B B/B/A | A/A/B | A/B/B B/A/A | A/B/B/B | B/A/B/B |
| B/B/B/A | B/B/A/B | A/A/B/B | A/B/A/B | A/B/B/A | B/B/A/A |
| B/A/B/A | B/A/A/B | A/A/A/B | B/A/A/A | A/B/A/A | A/A/B/A |

Administration of the composition MBP or peptide-mimic or analog to a cell, tissue or organism may follow general protocols for the administration of chemotherapeutics, taking into account the toxicity, if any. It is expected that the treatment cycles would be repeated as necessary. In particular embodiments, it is contemplated that various additional agents may be applied in any combination with the present invention.

1. Chemotherapeutic Agents

The term “chemotherapy” refers to the use of drugs to treat cancer. A “chemotherapeutic agent” is used to connote a compound or composition that is administered in the treatment of cancer. One subtype of chemotherapy known as biochemotherapy involves the combination of a chemotherapy with a biological therapy.

Chemotherapeutic agents include, but are not limited to, 5-fluorouracil, bleomycin, busulfan, camptothecin, carboplatin, chlorambucil, cisplatin (CDDP), cyclophosphamide, dactinomycin, daunorubicin, doxorubicin, estrogen receptor binding agents, etoposide (VP16), farnesyl-protein transferase inhibitors, gemcitabine, ifosfamide, mechlorethamine, melphalan, mitomycin, navelbine, nitrosurea, plicomycin, procarbazine, raloxifene, tamoxifen, taxol, temazolomide (an aqueous form of DTIC), transplatinum, vinblastine and methotrexate, vincristine, or any analog or derivative variant of the foregoing. These agents or drugs are categorized by their mode of activity within a cell, for example, whether and at what stage they affect the cell cycle. Alternatively, an agent may be characterized based on its ability to directly cross-link DNA, to intercalate into DNA, or to induce chromosomal and mitotic aberrations by affecting nucleic acid synthesis. Most chemotherapeutic agents fall into the following categories: alkylating agents, antimetabolites, antitumor antibiotics, corticosteroid hormones, mitotic inhibitors, and nitrosoureas, hormone agents, miscellaneous agents, and any analog or derivative variant thereof.

Chemotherapeutic agents and methods of administration, dosages, etc. are well known to those of skill in the art (see for example, the “Physicians Desk Reference”, Goodman & Gilman's “The Pharmacological Basis of Therapeutics”, “Remington's Pharmaceutical Sciences”, and “The Merck Index, Eleventh Edition”, incorporated herein by reference in relevant parts), and may be combined with the invention in light of the disclosures herein. Some variation in dosage will necessarily occur depending on the condition of the subject being treated. The person responsible for administration will, in any event, determine the appropriate dose for the individual subject. Examples of specific chemotherapeutic agents and dose regimes are also described herein. Of course, all of these dosages and agents described herein are exemplary rather than limiting, and other doses or agents may be used by a skilled artisan for a specific patient or application. Any dosage in-between these points, or range derivable therein is also expected to be of use in the invention.

a. Alkylating agents

Alkylating agents are drugs that directly interact with genomic DNA to prevent the cancer cell from proliferating. This category of chemotherapeutic drugs represents agents that affect all phases of the cell cycle, that is, they are not phase-specific. Alkylating agents can be implemented to treat, for example, chronic leukemia, non-Hodgkin's lymphoma, Hodgkin's disease, multiple myeloma, and particular cancers of the breast, lung, and ovary. An alkylating agent, may include, but is not limited to, a nitrogen mustard, an ethylenimene, a methylmelamine, an alkyl sulfonate, a nitrosourea or a triazines.

They include but are not limited to: busulfan, chlorambucil, cisplatin, cyclophosphamide (cytoxan), dacarbazine, ifosfamide, mechlorethamine (mustargen), and melphalan. In specific aspects, troglitazaone can be used to treat cancer in combination with any one or more of these alkylating agents, some of which are discussed below.

i. Nitrogen Mustards

A nitrogen mustard may be, but is not limited to, mechlorethamine (HN₂), which is used for Hodgkin's disease and non-Hodgkin's lymphomas; cyclophosphamide and/or ifosfamide, which are used in treating such cancers as acute or chronic lymphocytic leukemias, Hodgkin's disease, non-Hodgkin's lymphomas, multiple myeloma, neuroblastoma, breast, ovary, lung, Wilm's tumor, cervix testis and soft tissue sarcomas; melphalan (L-sarcolysin), which has been used to treat such cancers as multiple myeloma, breast and ovary; and chlorambucil, which has been used to treat diseases such as, for example, chronic lymphatic (lymphocytic) leukemia, malignant lymphomas including lymphosarcoma, giant follicular lymphoma, Hodgkin's disease and non-Hodgkin's lymphomas.

Chlorambucil (also known as leukeran) is a bifunctional alkylating agent of the nitrogen mustard type that has been found active against selected human neoplastic diseases.

Chlorambucil is known chemically as 4-[bis(2-chlorethyl)amino] benzenebutanoic acid.

Chlorambucil is available in tablet form for oral administration. It is rapidly and completely absorbed from the gastrointestinal tract. For example, after a single oral doses of about 0.6 mg/kg to about 1.2 mg/kg, peak plasma chlorambucil levels are reached within one hour and the terminal half-life of the parent drug is estimated at about 1.5 hours. About 0.1 mg/kg/day to about 0.2 mg/kg/day or about 3 6 mg/m²/day to about 6 mg/m²/day or alternatively about 0.4 mg/kg may be used for antineoplastic treatment. Chlorambucil is not curative by itself but may produce clinically useful palliation.

Cyclophosphamide is 2H-1,3,2-Oxazaphosphorin-2-amine, N,N-bis(2-chloroethyl)tetrahydro-, 2-oxide, monohydrate; termed Cytoxan available from Mead Johnson; and Neosar available from Adria. Cyclophosphamide is prepared by condensing 3-amino-1-propanol with N,N-bis(2-chlorethyl) phosphoramidic dichloride [(ClCH₂CH₂)₂N—POCl₂] in dioxane solution under the catalytic influence of triethylamine. The condensation is double, involving both the hydroxyl and the amino groups, thus effecting the cyclization.

Unlike other β-chloroethylamino alkylators, it does not cyclize readily to the active ethyleneimonium form until activated by hepatic enzymes. Thus, the substance is stable in the gastrointestinal tract, tolerated well and effective by the oral and parental routes and does not cause local vesication, necrosis, phlebitis or even pain.

Suitable oral doses for adults include, for example, about 1 mg/kg/day to about 5 mg/kg/day (usually in combination), depending upon gastrointestinal tolerance; or about 1 mg/kg/day to about 2 mg/kg/day; intravenous doses include, for example, initially about 40 mg/kg to about 50 mg/kg in divided doses over a period of about 2 days to about 5 days or about 10 mg/kg to about 15 mg/kg about every 7 days to about 10 days or about 3 mg/kg to about 5 mg/kg twice a week or about 1.5 mg/kg/day to about 3 mg/kg/day. In some aspects, a dose of about 250 mg/kg/day may be administered as an antineoplastic. Because of gastrointestinal adverse effects, the intravenous route is preferred for loading. During maintenance, a leukocyte count of about 3000/mm³ to 4000/mm³ usually is desired. The drug also sometimes is administered intramuscularly, by infiltration or into body cavities. It is available in dosage forms for injection of about 100 mg, about 200 mg and about 500 mg, and tablets of about 25 mg and about 50 mg.

Melphalan, also known as alkeran, L-phenylalanine mustard, phenylalanine mustard, L-PAM, or L-sarcolysin, is a phenylalanine derivative of nitrogen mustard. Melphalan is a bifunctional alkylating agent which is active against selective human neoplastic diseases. It is known chemically as 4-[bis(2-chloroethyl)amino]-L-phenylalanine.

Melphalan is the active L-isomer of the compound and was first synthesized in 1953 by Bergel and Stock; the D-isomer, known as medphalan, is less active against certain animal tumors, and the dose needed to produce effects on chromosomes is larger than that required with the L-isomer. The racemic (DL−) form is known as merphalan or sarcolysin. Melphalan is insoluble in water and has a pKa₁ of about 2.1. Melphalan is available in tablet form for oral administration and has been used to treat multiple myeloma. Available evidence suggests that about one third to one half of the patients with multiple myeloma show a favorable response to oral administration of the drug.

Melphalan has been used in the treatment of epithelial ovarian carcinoma. One commonly employed regimen for the treatment of ovarian carcinoma has been to administer melphalan at a dose of about 0.2 mg/kg daily for five days as a single course. Courses are repeated about every four to five weeks depending upon hematologic tolerance (Smith and Rutledge, 1975; Young et al., 1978). Alternatively in certain embodiments, the dose of melphalan used could be as low as about 0.05 mg/kg/day or as high as about 3 mg/kg/day or greater.

ii. Ethylenimenes and Methymelamines

An ethylenimene and/or a methylmelamine include, but are not limited to, hexamethylmelamine, used to treat ovary cancer; and thiotepa, which has been used to treat bladder, breast and ovary cancer.

iii. Alkyl Sulfonates

An alkyl sulfonate includes but is not limited to such drugs as busulfan, which has been used to treat chronic granulocytic leukemia.

Busulfan (also known as myleran) is a bifunctional alkylating agent. Busulfan is known chemically as 1,4-butanediol dimethanesulfonate. Busulfan is available in tablet form for oral administration, wherein for example, each scored tablet contains about 2 mg busulfan and the inactive ingredients magnesium stearate and sodium chloride.

Busulfan is indicated for the palliative treatment of chronic myelogenous (myeloid, myelocytic, granulocytic) leukemia. Although not curative, busulfan reduces the total granulocyte mass, relieves symptoms of the disease, and improves the clinical state of the patient. Approximately 90% of adults with previously untreated chronic myelogenous leukemia will obtain hematologic remission with regression or stabilization of organomegaly following the use of busulfan. Busulfan has been shown to be superior to splenic irradiation with respect to survival times and maintenance of hemoglobin levels, and to be equivalent to irradiation at controlling splenomegaly.

iv. Nitrosourea

Nitrosureas, like alkylating agents, inhibit DNA repair proteins. They are used to treat non-Hodgkin's lymphomas, multiple myeloma, malignant melanoma, in addition to brain tumors.

A nitrosourea include but is not limited to a carmustine (BCNU), a lomustine (CCNU), a semustine (methyl-CCNU) or a streptozocin. Semustine has been used in such cancers as a primary brain tumor, a stomach or a colon cancer. Stroptozocin has been used to treat diseases such as a malignant pancreatic insulinoma or a malignalnt carcinoid. Streptozocin has beeen used to treat such cancers as a malignant melanoma, Hodgkin's disease and soft tissue sarcomas.

Carmustine (sterile carmustine) is one of the nitrosoureas used in the treatment of certain neoplastic diseases. It is 1,3 bis (2-chloroethyl)-1-nitrosourea. It is lyophilized pale yellow flakes or congealed mass with a molecular weight of 214.06. It is highly soluble in alcohol and lipids, and poorly soluble in water. Carmustine is administered by intravenous infusion after reconstitution as recommended

Although it is generally agreed that carmustine alkylates DNA and RNA, it is not cross resistant with other alkylators. As with other nitrosoureas, it may also inhibit several key enzymatic processes by carbamoylation of amino acids in proteins.

Carmustine is indicated as palliative therapy as a single agent or in established combination therapy with other approved chemotherapeutic agents in brain tumors such as glioblastoma, brainstem glioma, medullobladyoma, astrocytoma, ependymoma, and metastatic brain tumors. Also it has been used in combination with prednisone to treat multiple myeloma. Carmustine has been used in treating such cancers as a multiple myeloma or a malignant melanoma. Carmustine has proved useful, in the treatment of Hodgkin's Disease and in non-Hodgkin's lymphomas, as secondary therapy in combination with other approved drugs in patients who relapse while being treated with primary therapy, or who fail to respond to primary therapy.

Sterile carmustine is commonly available in 100 mg single dose vials of lyophilized material. The recommended dose of carmustine as a single agent in previously untreated patients is about 150 mg/m² to about 200 mg/m² intravenously every 6 weeks. This may be given as a single dose or divided into daily injections such as about 75 mg/m² to about 100 mg/m² on 2 successive days. When carmustine is used in combination with other myelosuppressive drugs or in patients in whom bone marrow reserve is depleted, the doses should be adjusted accordingly. Doses subsequent to the initial dose should be adjusted according to the hematologic response of the patient to the preceding dose. It is of course understood that other doses may be used in the present invention, for example about 10 mg/m², about 20 mg/m², about 30 mg/m², about 40 mg/m², about 50 mg/m², about 60 mg/m², about 70 mg/m², about 80 mg/m², about 90 mg/m² to about 100 mg/m².

Lomustine is one of the nitrosoureas used in the treatment of certain neoplastic diseases. It is 1-(2-chloro-ethyl)-3-cyclohexyl-1 nitrosourea. It is a yellow powder with the empirical formula of C₉H₁₆ClN₃O₂ and a molecular weight of 233.71. Lomustine is soluble in 10% ethanol (about 0.05 mg/mL) and in absolute alcohol (about 70 mg/mL). Lomustine is relatively insoluble in water (less than about 0.05 mg/mL). It is relatively unionized at a physiological pH. Inactive ingredients in lomustine capsules are: magnesium stearate and mannitol.

Although it is generally agreed that lomustine alkylates DNA and RNA, it is not cross resistant with other alkylators. As with other nitrosoureas, it may also inhibit several key enzymatic processes by carbamoylation of amino acids in proteins.

Lomustine may be given orally. Following oral administration of radioactive lomustine at doses ranging from about 30 mg/m² to 100 mg/m², about half of the radioactivity given was excreted in the form of degradation products within 24 hours. The serum half-life of the metabolites ranges from about 16 hours to about 2 days. Tissue levels are comparable to plasma levels at 15 minutes after intravenous administration.

Lomustine has been shown to be useful as a single agent in addition to other treatment modalities, or in established combination therapy with other approved chemotherapeutic agents in both primary and metastatic brain tumors, in patients who have already received appropriate surgical and/or radiotherapeutic procedures. Lomustine has been used to treat such cancers as small-cell lung cancer. It has also proved effective in secondary therapy against Hodgkin's Disease in combination with other approved drugs in patients who relapse while being treated with primary therapy, or who fail to respond to primary therapy.

The recommended dose of lomustine in adults and children as a single agent in previously untreated patients is about 130 mg/m² as a single oral dose every 6 weeks. In individuals with compromised bone marrow function, the dose should be reduced to about 100 mg/m² every 6 weeks. When lomustine is used in combination with other myelosuppressive drugs, the doses should be adjusted accordingly. It is understood that other doses may be used for example, about 20 mg/m², about 30 mg/m², about 40 mg/m², about 50 mg/m², about 60 mg/m², about 70 mg/m², about 80 mg/m², about 90 mg/m², about 100 mg/m² to about 120 mg/m².

A triazine include but is not limited to such drugs as a dacabazine (DTIC; dimethyltriazenoimidaz olecarboxamide), used in the treatment of such cancers as a malignant melanoma, Hodgkin's disease and a soft-tissue sarcoma.

b. Antimetabolites

Antimetabolites disrupt DNA and RNA synthesis. Unlike alkylating agents, they specifically influence the cell cycle during S phase. They have used to combat chronic leukemias in addition to tumors of breast, ovary and the gastrointestinal tract. Antimetabolites can be differentiated into various categories, such as folic acid analogs, pyrimidine analogs and purine analogs and related inhibitory compounds. Antimetabolites include but are not limited to, 5-fluorouracil (5-FU), cytarabine (Ara-C), fludarabine, gemcitabine, and methotrexate.

i. Folic Acid Analogs

Folic acid analogs include but are not limited to compounds such as methotrexate (amethopterin), which has been used in the treatment of cancers such as acute lymphocytic leukemia, choriocarcinoma, mycosis fungoides, breast, head and neck, lung and osteogenic sarcoma.

ii. Pyrimidine Analogs

Pyrimidine analogs include such compounds as cytarabine (cytosine arabinoside), 5-fluorouracil (fluouracil; 5-FU) and floxuridine (fluorode-oxyuridine; FudR). Cytarabine has been used in the treatment of cancers such as acute granulocytic leukemia and acute lymphocytic leukemias. Floxuridine and 5-fluorouracil have been used in the treatment of cancers such as breast, colon, stomach, pancreas, ovary, head and neck, urinary bladder and topical premalignant skin lesions.

5-Fluorouracil (5-FU) has the chemical name of 5-fluoro-2,4(1H,3H)-pyrimidinedione. Its mechanism of action is thought to be by blocking the methylation reaction of deoxyuridylic acid to thymidylic acid. Thus, 5-FU interferes with the synthesis of deoxyribonucleic acid (DNA) and to a lesser extent inhibits the formation of ribonucleic acid (RNA). Since DNA and RNA are essential for cell division and proliferation, it is thought that the effect of 5-FU is to create a thymidine deficiency leading to cell death. Thus, the effect of 5-FU is found in cells that rapidly divide, a characteristic of metastatic cancers.

iii. Purine Analogs and Related Inhibitors

Purine analogs and related compounds include, but are not limited to, mercaptopurine (6-mercaptopurine; 6-MP), thioguanine (6-thioguanine; TG) and pentostatin (2-deoxycoformycin). Mercaptopurine has been used in acute lymphocytic, acute granulocytic and chronic granulocytic leukemias. Thrioguanine has been used in the treatment of such cancers as acute granulocytic leukemia, acute lymphocytic leukemia and chronic lymphocytic leukemia. Pentostatin has been used in such cancers as hairy cell leukemias, mycosis fungoides and chronic lymphocytic leukemia.

C. Natural Products

Natural products generally refer to compounds originally isolated from a natural source, and identified has having a pharmacological activity. Such compounds, analogs and derivatives thereof may be, isolated from a natural source, chemically synthesized or recombinantly produced by any technique known to those of skill in the art. Natural products include such categories as mitotic inhibitors, antitumor antibiotics, enzymes and biological response modifiers.

i. Mitotic Inhibitors

Mitotic inhibitors include plant alkaloids and other natural agents that can inhibit either protein synthesis required for cell division or mitosis. They operate during a specific phase during the cell cycle. Mitotic inhibitors include, for example, docetaxel, etoposide (VP16), teniposide, paclitaxel, taxol, vinblastine, vincristine, and vinorelbine.

Epipodophyllotoxins include such compounds as teniposide and VP16. VP16 is also known as etoposide and is used primarily for treatment of testicular tumors, in combination with bleomycin and cisplatin, and in combination with cisplatin for small-cell carcinoma of the lung. Teniposide and VP16 are also active against cancers such as testis, other lung cancer, Hodgkin's disease, non-Hodgkin's lymphomas, acute granulocytic leukemia, acute nonlymphocytic leukemia, carcinoma of the breast, and Kaposi's sarcoma associated with acquired immunodeficiency syndrome (AIDS).

VP16 is available as a solution (e.g., 20 mg/ml) for intravenous administration and as 50 mg, liquid-filled capsules for oral use. For small-cell carcinoma of the lung, the intravenous dose (in combination therapy) is can be as much as about 100 mg/m² or as little as about 2 mg/m², routinely about 35 mg/m², daily for about 4 days, to about 50 mg/m², daily for about 5 days have also been used. When given orally, the dose should be doubled. Hence the doses for small cell lung carcinoma may be as high as about 200 mg/m² to about 250 mg/m². The intravenous dose for testicular cancer (in combination therapy) is about 50 mg/m² to about 100 mg/m² daily for about 5 days, or about 100 mg/m² on alternate days, for three doses. Cycles of therapy are usually repeated about every 3 to 4 weeks. The drug should be administered slowly (e.g., about 30 minutes to about 60 minutes) as an infusion in order to avoid hypotension and bronchospasm, which are probably due to the solvents used in the formulation.

Taxoids are a class of related compounds isolated from the bark of the ash tree, Taxus brevifolia. Taxoids include but are not limited to compounds such as docetaxel and paclitaxel.

Paclitaxel binds to tubulin (at a site distinct from that used by the vinca alkaloids) and promotes the assembly of microtubules. Paclitaxel is being evaluated clinically; it has activity against malignant melanoma and carcinoma of the ovary. In certain aspects, maximal doses are about 30 mg/m² per day for about 5 days or about 210 mg/m² to about 250 mg/m² given once about every 3 weeks.

Vinca alkaloids are a type of plant alkaloid identified to have pharmaceutical activity. They include such compounds as vinblastine (VLB) and vincristine.

Vinblastine is an example of a plant alkaloid that can be used for the treatment of cancer and precancer. When cells are incubated with vinblastine, dissolution of the microtubules occurs.

Unpredictable absorption has been reported after oral administration of vinblastine or vincristine. At the usual clinical doses the peak concentration of each drug in plasma is approximately 0.4 mM. Vinblastine and vincristine bind to plasma proteins. They are extensively concentrated in platelets and to a lesser extent in leukocytes and erythrocytes.

After intravenous injection, vinblastine has a multiphasic pattern of clearance from the plasma; after distribution, drug disappears from plasma with half-lives of approximately 1 and 20 hours. Vinblastine is metabolized in the liver to biologically activate derivative desacetylvinblastine. Approximately 15% of an administered dose is detected intact in the urine, and about 10% is recovered in the feces after biliary excretion. Doses should be reduced in patients with hepatic dysfunction. At least a 50% reduction in dosage is indicated if the concentration of bilirubin in plasma is greater than 3 mg/dl (about 50 mM).

Vinblastine sulfate is available in preparations for injection. When the drug is given intravenously; special precautions must be taken against subcutaneous extravasation, since this may cause painful irritation and ulceration. The drug should not be injected into an extremity with impaired circulation. After a single dose of 0.3 mg/kg of body weight, myelosuppression reaches its maximum in about 7 days to about 10 days. If a moderate level of leukopenia (approximately 3000 cells/mm³) is not attained, the weekly dose may be increased gradually by increments of about 0.05 mg/kg of body weight. In regimens designed to cure testicular cancer, vinblastine is used in doses of about 0.3 mg/kg about every 3 weeks irrespective of blood cell counts or toxicity.

An important clinical use of vinblastine is with bleomycin and cisplatin in the curative therapy of metastatic testicular tumors. Beneficial responses have been reported in various lymphomas, particularly Hodgkin's disease, where significant improvement may be noted in 50 to 90% of cases. The effectiveness of vinblastine in a high proportion of lymphomas is not diminished when the disease is refractory to alkylating agents. It is also active in Kaposi's sarcoma, testis cancer, neuroblastoma, and Letterer-Siwe disease (histiocytosis X), as well as in carcinoma of the breast and choriocarcinoma in women.

Doses of about 0.1 mg/kg to about 0.3 mg/kg can be administered or about 1.5 mg/m² to about 2 mg/m² can also be administered. Alternatively, about 0.1 mg/m², about 0.12 mg/m², about 0.14 mg/m², about 0.15 mg/m², about 0.2 mg/m², about 0.25 mg/m², about 0.5 mg/m², about 1.0 mg/m², about 1.2 mg/m², about 1.4 mg/m², about 1.5 mg/m², about 2.0 mg/m², about 2.5 mg/m², about 5.0 mg/m², about 6 mg/m², about 8 mg/m², about 9 mg/m², about 10 mg/m² to about 20 mg/m², can be given.

Vincristine blocks mitosis and produces metaphase arrest. It seems likely that most of the biological activities of this drug can be explained by its ability to bind specifically to tubulin and to block the ability of protein to polymerize into microtubules. Through disruption of the microtubules of the mitotic apparatus, cell division is arrested in metaphase. The inability to segregate chromosomes correctly during mitosis presumably leads to cell death.

The relatively low toxicity of vincristine for normal marrow cells and epithelial cells make this agent unusual among anti-neoplastic drugs, and it is often included in combination with other myelosuppressive agents.

Unpredictable absorption has been reported after oral administration of vinblastine or vincristine. At the usual clinical doses the peak concentration of each drug in plasma is about 0.4 mM.

Vinblastine and vincristine bind to plasma proteins. They are extensively concentrated in platelets and to a lesser extent in leukocytes and erythrocytes. Vincristine has a multiphasic pattern of clearance from the plasma; the terminal half-life is about 24 hours. The drug is metabolized in the liver, but no biologically active derivatives have been identified. Doses should be reduced in patients with hepatic dysfunction. At least a 50% reduction in dosage is indicated if the concentration of bilirubin in plasma is greater than about 3 mg/dl (about 50 mM).

Vincristine sulfate is available as a solution (e.g., 1 mg/ml) for intravenous injection. Vincristine used together with corticosteroids is presently the treatment of choice to induce remissions in childhood leukemia; the optimal dosages for these drugs appear to be vincristine, intravenously, about 2 mg/m² of body-surface area, weekly; and prednisone, orally, about 40 mg/m², daily. Adult patients with Hodgkin's disease or non-Hodgkin's lymphomas usually receive vincristine as a part of a complex protocol. When used in the MOPP regimen, the recommended dose of vincristine is about 1.4 mg/m². High doses of vincristine seem to be tolerated better by children with leukemia than by adults, who may experience sever neurological toxicity. Administration of the drug more frequently than every 7 days or at higher doses seems to increase the toxic manifestations without proportional improvement in the response rate.

Precautions should also be used to avoid extravasation during intravenous administration of vincristine. Vincristine (and vinblastine) can be infused into the arterial blood supply of tumors in doses several times larger than those that can be administered intravenously with comparable toxicity.

Vincristine has been effective in Hodgkin's disease and other lymphomas. Although it appears to be somewhat less beneficial than vinblastine when used alone in Hodgkin's disease, when used with mechlorethamine, prednisone, and procarbazine (the so-called MOPP regimen), it is the preferred treatment for the advanced stages (III and IV) of this disease. In non-Hodgkin's lymphomas, vincristine is an important agent, particularly when used with cyclophosphamide, bleomycin, doxorubicin, and prednisone. Vincristine is more useful than vinblastine in lymphocytic leukemia. Beneficial response have been reported in patients with a variety of other neoplasms, particularly Wilms' tumor, neuroblastoma, brain tumors, rhabdomyosarcoma, small cell lung, and carcinomas of the breast, bladder, and the male and female reproductive systems.

Doses of vincristine include about 0.01 mg/kg to about 0.03 mg/kg or about 0.4 mg/m² to about 1.4 mg/m² can be administered or about 1.5 mg/m² to about 2 mg/m² can also be administered. Alternatively, in certain embodiments, about 0.02 mg/m², about 0.05 mg/m², about 0.06 mg/m², about 0.07 mg/m², about 0.08 mg/m², about 0.1 mg/m², about 0.12 mg/m², about 0.14 mg/m², about 0.15 mg/m², about 0.2 mg/m², about 0.25 mg/m² can be given as a constant intravenous infusion.

d. Antitumor Antibiotics

Antitumor antibiotics have both antimicrobial and cytotoxic activity. These drugs also interfere with DNA by chemically inhibiting enzymes and mitosis or altering cellular membranes. These agents are not phase specific so they work in all phases of the cell cycle. Thus, they are widely used for a variety of cancers. Examples of antitumor antibiotics include, but are not limited to, bleomycin, dactinomycin, daunorubicin, doxorubicin (Adriamycin), plicamycin (mithramycin) and idarubicin. Widely used in clinical setting for the treatment of neoplasms these compounds generally are administered through intravenous bolus injections or orally.

Doxorubicin hydrochloride, 5,12-Naphthacenedione, (8s-cis)-10-[(3-amino-2,3,6-trideoxy-a-L-lyxo-hexopyranosyl)oxy]-7,8,9,10-tetrahydro-6,8,11-trihydroxy-8-(hydroxyacetyl)-1-methoxy-hydrochloride (hydroxydaunorubicin hydrochloride, Adriamycin) is used in a wide antineoplastic spectrum. It binds to DNA and inhibits nucleic acid synthesis, inhibits mitosis and promotes chromosomal aberrations.

Administered alone, it is the drug of first choice for the treatment of thyroid adenoma and primary hepatocellular carcinoma. It is a component of 31 first-choice combinations for the treatment of diseases including ovarian, endometrial and breast tumors, bronchogenic oat-cell carcinoma, non-small cell lung carcinoma, stomach, genitourinary, thyroid, gastric adenocarcinoma, retinoblastoma, neuroblastoma, mycosis fungoides, pancreatic carcinoma, prostatic carcinoma, bladder carcinoma, myeloma, diffuse histiocytic lymphoma, Wilns' tumor, Hodgkin's disease, adrenal tumors, osteogenic sarcoma, soft tissue sarcoma, Ewing's sarcoma, rhabdomyosarcoma and acute lymphocytic leukemia. It is an alternative drug for the treatment of other diseases such as islet cell, cervical, testicular and adrenocortical cancers. It is also an immunosuppressant.

Doxorubicin is absorbed poorly and is preferably administered intravenously. The pharmacokinetics are multicompartmental. Distribution phases have half-lives of 12 minutes and 3.3 hours. The elimination half-life is about 30 hours, with about 40% to about 50% secreted into the bile. Most of the remainder is metabolized in the liver, partly to an active metabolite (doxorubicinol), but a few percent is excreted into the urine. In the presence of liver impairment, the dose should be reduced.

In certain embodiments, appropriate intravenous doses are, adult, about 60 mg/m² to about 75 mg/m² at about 21-day intervals or about 25 mg/m² to about 30 mg/m² on each of 2 or 3 successive days repeated at about 3 week to about 4 week intervals or about 20 mg/m² once a week. The lowest dose should be used in elderly patients, when there is prior bone-marrow depression caused by prior chemotherapy or neoplastic marrow invasion, or when the drug is combined with other myelopoietic suppressant drugs. The dose should be reduced by about 50% if the serum bilirubin lies between about 1.2 mg/dL and about 3 mg/dL and by about 75% if above about 3 mg/dL. The lifetime total dose should not exceed about 550 mg/m² in patients with normal heart function and about 400 mg/m² in persons having received mediastinal irradiation. In certain embodiments, and alternative dose regiment may comprise about 30 mg/m² on each of 3 consecutive days, repeated about every 4 week. Exemplary doses may be about 10 mg/m², about 20 mg/m², about 30 mg/m², about 50 mg/m², about 100 mg/m², about 150 mg/m², about 175 mg/m², about 200 mg/m², about 225 mg/m², about 250 mg/m², about 275 mg/m², about 300 mg/m², about 350 mg/m², about 400 mg/m², about 425 mg/m², about 450 mg/m², about 475 mg/m², to about 500 mg/m².

Daunorubicin hydrochloride, 5,12-Naphthacenedione, (8S-cis)-8-acetyl-10-[(3-amino-2,3,6-trideoxy-a-L-lyxo-hexanopyranosyl)oxy]-7,8,9,10-tetrahydro-6,8,11-trihydroxy-10-methoxy-, hydrochloride; also termed cerubidine and available from Wyeth. Daunorubicin (daunomycin; rubidomycin) intercalates into DNA, blocks DAN-directed RNA polymerase and inhibits DNA synthesis. It can prevent cell division in doses that do not interfere with nucleic acid synthesis.

In combination with other drugs it is often included in the first-choice chemotherapy of diseases such as, for example, acute granulocytic leukemia, acute myelocytic leukemia in adults (for induction of remission), acute lymphocytic leukemia and the acute phase of chronic myelocytic leukemia. Oral absorption is poor, and it preferably given by other methods (e.g., intravenously). The half-life of distribution is 45 minutes and of elimination, about 19 hours.

The half-life of its active metabolite, daunorubicinol, is about 27 hours. Daunorubicin is metabolized mostly in the liver and also secreted into the bile (about 40%). Dosage must be reduced in liver or renal insufficiencies.

Generally, suitable intravenous doses are (base equivalent): adult, younger than 60 years, about 45 mg/m²/day (about 30 mg/m² for patients older than 60 year.) for about 1 day, about 2 days or about 3 days about every 3 weeks or 4 weeks or about 0.8 mg/kg/day for about 3 days, about 4 days, about 5 days to about 6 days about every 3 weeks or about 4 weeks; no more than about 550 mg/m² should be given in a lifetime, except only about 450 mg/m² if there has been chest irradiation; children, about 25 mg/m² once a week unless the age is less than 2 years. or the body surface less than about 0.5 m, in which case the weight-based adult schedule is used. It is available in injectable dosage forms (base equivalent) of about 20 mg (as the base equivalent to about 21.4 mg of the hydrochloride). Exemplary doses may be about 10 mg/m², about 20 mg/m², about 30 mg/m², about 50 mg/m², about 100 mg/m², about 150 mg/m², about 175 mg/m², about 200 mg/m², about 225 mg/m², about 250 mg/m², about 275 mg/m², about 300 mg/m², about 350 mg/m², about 400 mg/m², about 425 mg/m², about 450 mg/m², about 475 mg/m², to about 500 mg/m².

Mitomycin (also known as mutamycin and/or mitomycin-C) is an antibiotic isolated from the broth of Streptomyces caespitosus which has been shown to have antitumor activity. The compound is heat stable, has a high melting point, and is freely soluble in organic solvents.

Mitomycin selectively inhibits the synthesis of deoxyribonucleic acid (DNA). The guanine and cytosine content correlates with the degree of mitomycin-induced cross-linking. At high concentrations of the drug, cellular RNA and protein synthesis are also suppressed. Mitomycin has been used in tumors such as stomach, cervix, colon, breast, pancreas, bladder and head and neck.

In humans, mitomycin is rapidly cleared from the serum after intravenous administration.

Time required to reduce the serum concentration by about 50% after a 30 mg. bolus injection is 17 minutes. After injection of 30 mg, 20 mg, or 10 mg I.V., the maximal serum concentrations were 2.4 mg/mL, 1.7 mg/mL, and 0.52 mg/mL, respectively. Clearance is effected primarily by metabolism in the liver, but metabolism occurs in other tissues as well. The rate of clearance is inversely proportional to the maximal serum concentration because, it is thought, of saturation of the degradative pathways. Approximately 10% of a dose of mitomycin is excreted unchanged in the urine. Since metabolic pathways are saturated at relatively low doses, the percent of a dose excreted in urine increases with increasing dose. In children, excretion of intravenously administered mitomycin is similar.

Actinomycin D (Dactinomycin) [50-76-0]; C₆₂H₈₆N₁₂O₁₆ (1255.43) is an antineoplastic drug that inhibits DNA-dependent RNA polymerase. It is often a component of first-choice combinations for treatment of diseases such as, for example, choriocarcinoma, embryonal rhabdomyosarcoma, testicular tumor, Kaposi's sarcoma and Wilms' tumor. Tumors that fail to respond to systemic treatment sometimes respond to local perfusion. Dactinomycin potentiates radiotherapy. It is a secondary (efferent) immunosuppressive.

In certain specific aspects, actinomycin D is used in combination with agents such as, for example, primary surgery, radiotherapy, and other drugs, particularly vincristine and cyclophosphamide. Antineoplastic activity has also been noted in Ewing's tumor, Kaposi's sarcoma, and soft-tissue sarcomas. Dactinomycin can be effective in women with advanced cases of choriocarcinoma. It also produces consistent responses in combination with chlorambucil and methotrexate in patients with metastatic testicular carcinomas. A response may sometimes be observed in patients with Hodgkin's disease and non-Hodgkin's lymphomas. Dactinomycin has also been used to inhibit immunological responses, particularly the rejection of renal transplants.

Half of the dose is excreted intact into the bile and 10% into the urine; the half-life is about 36 hours. The drug does not pass the blood-brain barrier. Actinomycin D is supplied as a lyophilized powder (0/5 mg in each vial). The usual daily dose is about 10 mg/kg to about 15 mg/kg; this is given intravenously for about 5 days; if no manifestations of toxicity are encountered, additional courses may be given at intervals of about 3 weeks to about 4 weeks. Daily injections of about 100 mg to about 400 mg have been given to children for about 10 days to about 14 days; in other regimens, about 3 mg/kg to about 6 mg/kg, for a total of about 125 mg/kg, and weekly maintenance doses of about 7.5 mg/kg have been used. Although it is safer to administer the drug into the tubing of an intravenous infusion, direct intravenous injections have been given, with the precaution of discarding the needle used to withdraw the drug from the vial in order to avoid subcutaneous reaction. Exemplary doses may be about 100 mg/m², about 150 mg/m², about 175 mg/m², about 200 mg/m², about 225 mg/m², about 250 mg/m², about 275 mg/m², about 300 mg/m², about 350 mg/m², about 400 mg/m², about 425 mg/m², about 450 mg/m², about 475 mg/m², to about 500 mg/m².

Bleomycin is a mixture of cytotoxic glycopeptide antibiotics isolated from a strain of Streptomyces verticillus. Although the exact mechanism of action of bleomycin is unknown, available evidence would seem to indicate that the main mode of action is the inhibition of DNA synthesis with some evidence of lesser inhibition of RNA and protein synthesis.

In mice, high concentrations of bleomycin are found in the skin, lungs, kidneys, peritoneum, and lymphatics. Tumor cells of the skin and lungs have been found to have high concentrations of bleomycin in contrast to the low concentrations found in hematopoietic tissue. The low concentrations of bleomycin found in bone marrow may be related to high levels of bleomycin degradative enzymes found in that tissue.

In patients with a creatinine clearance of greater than about 35 mL per minute, the serum or plasma terminal elimination half-life of bleomycin is approximately 115 minutes. In patients with a creatinine clearance of less than about 35 mL per minute, the plasma or serum terminal elimination half-life increases exponentially as the creatinine clearance decreases. In humans, about 60% to about 70% of an administered dose is recovered in the urine as active bleomycin. In specific embodiments, bleomycin may be given by the intramuscular, intravenous, or subcutaneous routes. It is freely soluble in water. Because of the possibility of an anaphylactoid reaction, lymphoma patients should be treated with two units or less for the first two doses. If no acute reaction occurs, then the regular dosage schedule may be followed.

In preferred aspects, bleomycin should be considered a palliative treatment. It has been shown to be useful in the management of the following neoplasms either as a single agent or in proven combinations with other approved chemotherapeutic agents in squamous cell carcinoma such as head and neck (including mouth, tongue, tonsil, nasopharynx, oropharynx, sinus, palate, lip, buccal mucosa, gingiva, epiglottis, larynx), esophagus, lung and genitourinary tract, Hodgkin's disease, non-Hodgkin's lymphoma, skin, penis, cervix, and vulva. It has also been used in the treatment of lymphomas and testicular carcinoma.

Improvement of Hodgkin's Disease and testicular tumors is prompt and noted within 2 weeks. If no improvement is seen by this time, improvement is unlikely. Squamous cell cancers respond more slowly, sometimes requiring as long as 3 weeks before any improvement is noted.

2. Hormones and Antagonists

Hormonal therapy may also be used in conjunction with the present invention and/or in combination with any other cancer therapy or agent(s). The use of hormones may be employed in the treatment of certain cancers such as breast, prostate, ovarian, or cervical cancer to lower the level or block the effects of certain hormones such as testosterone or estrogen. This treatment is often used in combination with at least one other cancer therapy as a treatment option or to reduce the risk of metastases.

Corticosteroid hormones are useful in treating some types of cancer (e.g. non-Hodgkin's lymphoma, acute and chronic lymphocytic leukemias, breast cancer, and multiple myeloma). Though these hormones have been used in the treatment of many non-cancer conditions, they are considered chemotherapy drugs when they are implemented to kill or slow the growth of cancer cells. Corticosteroid hormones can increase the effectiveness of other chemotherapy agents, and consequently, they are frequently used in combination treatments. Prednisone and dexamethasone are examples of corticosteroid hormones.

Progestins such as hydroxyprogesterone caproate, medroxyprogesterone acetate, and megestrol acetate have been used in cancers of the endometrium and breast. Estrogens such as diethylstilbestrol and ethinyl estradiol have been used in cancers such as breast and prostate. Antiestrogens such as tamoxifen have been used in cancers such as breast. Androgens such as testosterone propionate and fluoxymesterone have also been used in treating breast cancer. Antiandrogens such as flutamide have been used in the treatment of prostate cancer. Gonadotropin-releasing hormone analogs such as leuprolide have been used in treating prostate cancer. U.S. Pat. No. 4,418,068, incorporated herein by reference, discloses antiestrogenic and antiandrogenic benzothiophenes, such as, for example, 6-hydroxy-2-(4-hydroxyphenyl)-3-[4-(2-piperidinoethoxy)benzoyl]benzo[b]thiophene, and esters, ethers, and salts thereof for the treatment of cancers such as prostate and breast cancer.

3. Miscellaneous Agents

Some chemotherapy agents do not qualify into the previous categories based on their activities. They include, but are not limited to, platinum coordination complexes, anthracenedione, substituted urea, methyl hydrazine derivative, adrenalcortical suppressant, amsacrine, L-asparaginase, and tretinoin. It is contemplated that they are included within the compositions and methods of the present invention for use in combination therapies.

Platinum coordination complexes include such compounds as carboplatin and cisplatin (cis-DDP). Cisplatin has been widely used to treat cancers such as, for example, metastatic testicular or ovarian carcinoma, advanced bladder cancer, head or neck cancer, cervical cancer, lung cancer or other tumors. Cisplatin is not absorbed orally and must therefore be delivered via other routes, such as for example, intravenous, subcutaneous, intratumoral or intraperitoneal injection. Cisplatin can be used alone or in combination with other agents, with efficacious doses used in clinical applications of about 15 mg/m² to about 20 mg/m² for 5 days every three weeks for a total of three courses being contemplated in certain embodiments. Doses may be, for example, about 0.50 mg/m², about 1.0 mg/m², about 1.50 mg/m², about 1.75 mg/m², about 2.0 mg/m², about 3.0 mg/m², about 4.0 mg/m², about 5.0 mg/m², to about 10 mg/m².An anthracenedione such as mitoxantrone has been used for treating acute granulocytic leukemia and breast cancer. A substituted urea such as hydroxyurea has been used in treating chronic granulocytic leukemia, polycythemia vera, essental thrombocytosis and malignant melanoma. A methyl hydrazine derivative such as procarbazine (N-methylhydrazine, MIH) has been used in the treatment of Hodgkin's disease. An adrenocortical suppressant such as mitotane has been used to treat adrenal cortex cancer, while aminoglutethimide has been used to treat Hodgkin's disease.

4. Radiotherapeutic Agents

Radiotherapeutic agents include radiation and waves that induce DNA damage for example, γ-irradiation, X-rays, proton beam therapies (U.S. Pat. Nos. 5,760,395 and 4,870,287), UV-irradiation, microwaves, electronic emissions, radioisotopes, and the like. Therapy may be achieved by irradiating the localized tumor site with the above described forms of radiations. It is most likely that all of these agents effect a broad range of damage DNA, on the precursors of DNA, the replication and repair of DNA, and the assembly and maintenance of chromosomes.

Radiotherapeutic agents and methods of administration, dosages, etc. are well known to those of skill in the art, and may be combined with the invention in light of the disclosures herein. For example, dosage ranges for X-rays range from daily doses of 50 to 200 roentgens for prolonged periods of time (3 to 4 weeks), to single doses of 2000 to 6000 roentgens. Dosage ranges for radioisotopes vary widely, and depend on the half-life of the isotope, the strength and type of radiation emitted, and the uptake by the neoplastic cells.

5. Immunotherapeutic Agents

An immunotherapeutic agent generally relies on the use of immune effector cells and molecules to target and destroy cancer cells. The immune effector may be, for example, an antibody specific for some marker on the surface of a tumor cell. The antibody alone may serve as an effector of therapy or it may recruit other cells to actually effect cell killing. The antibody also may be conjugated to a drug or toxin (e.g., a chemotherapeutic, a radionuclide, a ricin A chain, a cholera toxin, a pertussis toxin, etc.) and serve merely as a targeting agent. Such antibody conjugates are called immunotoxins, and are well known in the art (see U.S. Pat. Nos. 5,686,072, 5,578,706, 4,792,447, 5,045,451, 4,664,911, and 5,767,072, each incorporated herein by reference). Alternatively, the effector may be a lymphocyte carrying a surface molecule that interacts, either directly or indirectly, with a tumor cell target. Various effector cells include cytotoxic T cells and NK cells.

In one aspect of immunotherapy, the tumor cell must bear some marker that is amenable to targeting, i.e., is not present on the majority of other cells. Many tumor markers exist and any of these may be suitable for targeting in the context of the present invention. Common tumor markers include carcinoembryonic antigen, prostate specific antigen, urinary tumor associated antigen, fetal antigen, tyrosinase (p97), gp68, TAG-72, HMFG, Sialyl Lewis Antigen, MucA, MucB, PLAP, estrogen receptor, laminin receptor, erb B and p155.

### 8. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### NMR spectroscopy

A combination of the traditional homonuclear (¹H/¹H), heteronuclear double-(¹⁵N/¹H and ¹³C/¹H), and triple-resonance (¹⁵N/¹³C/¹H) NMR experiments are acquired on fully protonated, partially deuterated, or fully deuterated samples (reviewed in Bax et al., 1993; Clore et al., 1994; Clore et al., 1998; Clore et al., 1997; Gardner et al., 1998; Sattler et al., 1999; Nietlispach et al., 1996; Kay et al., 1997; Lin et al., 1999). All isotopic labels involve uniform, ca. 100% incorporation unless indicated otherwise next to the corresponding isotope. The software that is used for NMR data processing is NMRPipe (Delaglio et al., 1995), whereas NMR data analysis is performed with NMRView (Johnson et al., 1994) and structure calculations are performed with the program CNS (Brunger et al., 1998).

The protein backbone and Cβ resonance assignments were assigned primarily using HNCA, HN(CO)CA, HN(CA)CB and HN(COCA)CB experiments acquired on a ²H,¹⁵N,¹³C-Mad2-MBP1 sample. Partial Hoc and aliphatic side chain resonance assignment were also obtained from (H)C(CO)NH-TOCSY and H(C)(CO)NH-TOCSY experiments acquired on a 60%-²H,¹⁵N, ¹³C-Mad2-MBP1 sample. A more complete assignments of the side chain resonances is obtained through the analysis of HCCH-COSY, HCCH-TOCSY, and ¹H—¹³C NOESY-HSQC experiments acquired on a ¹H,¹³C-Mad2-MBP1 sample, and 3D ¹H—¹⁵N NOESY-HSQC and TOCSY-HSQC experiments acquired on a ¹⁵N-Mad2-MBP1 sample.

Aromatic side chain resonances are assigned from homonuclear 2D NOESY, TOCSY, and DQF-COSY experiments acquired on a ¹⁵N-Mad2-MBP1 sample, and aided by ¹H—¹³C NOESY-HSQC and ¹H—¹³C CT-HSQC experiments acquired on a ¹⁵N,¹³C-Mad2-MBP1 sample. The peptide resonances were assigned using 3D ¹H—¹⁵N NOESY-HSQC and TOCSY-HSQC experiments acquired on a Mad2¹⁵N-MBP1 sample, and HNCA, HN(CO)CA, ¹H—¹³C NOESY-HSQC and ¹H-—¹³C CT-HSQC experiments acquired on a Mad2-¹⁵N,¹³C-MBP1 sample. Stereospecific assignment of valine and leucine methyl groups of Mad2 are obtained from high resolution ¹H—¹³C CT-HSQC spectra acquired on 10% ¹³C-labeled samples (only for the Mad2 portion in the Mad2-MBP1 complex).

All the 2D and 3D NOESY data are used to assign intramolecular NOEs of either Mad2 or MBP1 for structure determination. Because of the fewer number of peptide resonances, some intermolecular NOEs are easily derived from the 3D ¹ H—¹⁵N NOESY-HSQC and 3D ¹H—¹³C NOESY-HSQC data acquired with the labeled peptide samples.

### Example 2

### Structure Determination

The NOE cross-peak intensities are calibrated against some internal standard fixed length and classified into three different categories with distances of 1.8-2.7 Å, 1.8-3.3 Å, and 1.8-5.0 Å for strong, medium, and weak NOEs, respectively. Protection of amide protons from exchange with the solvent is measured from the rate of disappearance of ¹H-5N HSQC cross-peaks after dissolving the protein in D₂O, and from the intensity of solvent exchange cross-peaks in 3D ¹H-¹⁵ N TOCSY-HSQC experiments. Amide protons that exchange slowly with solvent are usually involved in hydrogen bonds. In combination with the identifiable regular secondary structures, specific hydrogen bond restraints are obtained. The 3JHNα coupling constants are measured using HNHA spectra. The Φ and Ψ torsion angle restraints are derived from the measured 3JHNα coupling constants and analysis of backbone and Cβ chemical shifts using the program TALOS (Cornilescu et al., 1999). The restraints are a margin of twice the standard deviations observed in the TALOS database matches, with a minimum of 30°.

Interproton distance, hydrogen bond and torsion angle restraints deduced from all these measurements are incorporated progressively into simulated annealing calculations using torsional dynamics to obtain three-dimensional structures consistent with the NMR restraints (Brunger et al., 1998; Stein et al. 1997). The initial structure calculated from a conservative set of NOE and torsion angle restraints provides the starting point for structure refinement. Systematic restraint violations are checked against the data for possible errors in the assignment of NOE cross-peaks or in the category of restraints. Restraints are added when NOE assignment ambiguities are clearly resolved by the calculated structures. Assignment of ambiguous NOEs is aided by the program ARIA in combination with CNS (Nilges et al., 1997). Structure calculations are repeated with the revised and expanded restraints until a satisfactory set of structures are obtained and all restraints are met.

### Example 3

### Determination of Binding Regions of Mad2

Several backbone amide ¹H—¹⁵N HSQC signals of Cdc20₁₁₁₋₁₅₀ disappeared when bound to Mad2. The rest of the HSQC signals of Cdc20₁₁₁₋₁₅₀ did not undergo chemical shift changes between the free and the Mad2-bound forms.

Based on the 3D ¹⁵N-NOESY-HSQC and ¹⁵N-TOCSY-HSQC spectra acquired on the Mad2-¹⁵N-Cdc20₁₁₁₋₁₅₀ complex, the visible signals in the HSQC spectrum belonged to the N- and C-terminal residues of Cdc20₁₁₁₋₁₅₀. Therefore, only a small segment of Cdc20₁₁₁₋₁₅₀ was directly involved in binding to Mad2 and the backbone HSQC signals of these residues became invisible when bound to Mad2 presumably due to exchange processes with intermediate rates on the NMR time scale.

To optimize the Mad2-binding Cdc20 peptide, the inventors produced a 14-amino acid peptide, Cdc20P1, corresponding to residues 124-137 of Cdc20. Cdc20P1 did not contain the residues within Cdc20₁₁₁₋₁₅₀ that were not involved in binding to Mad2. The HSQC spectrum of the Mad2-¹⁵N-Cdc20P1 complex revealed that six or seven residues of Cdc20P1 interacted directly with Mad2. The backbone amide HSQC signals of these residues were visible although the intensities of these signals were weak. Interestingly, the amide proton chemical shifts of three residues in Cdc20P1 were below 9 ppm, suggesting that Cdc20P1 might adopt a P strand conformation when bound to Mad2.

Next, the inventors produced a H₂O sample of the Mad2-Cdc20P1 complex with the Mad2 protein labeled with ¹⁵N, ¹³C, and ²H. A series of 3D triple-resonance type of spectra, including HNCA, HN(CO)CA, HN(COCA)CB, and HN(CA)CB, were acquired. Analysis of these spectra revealed that there were two sets of resonances in these spectra: one set of signals was sharp and dispersed while the other set of signals was much broader and clustered. This suggested that the Mad2-Cdc20P1 complex might aggregate over time.

To confirm if the complex aggregated over time, an HSQC spectrum was acquired on a freshly prepared ¹⁵N-Mad2-Cdc20P1 sample. This sample was then incubated at 30° C. (the temperature for NMR data acquisition) for 48 hours and another HSQC was acquired. Comparison of the two HSQC spectra revealed that a set of broad signals emerged in the center of the spectrum after incubation. Therefore, the Mad2-Cdc20P1 complex slowly forms large molecular weight aggregates at ambient temperature, seriously hindering our structural analysis.

Binding affinity between Mad2 and Cdc20P1 was determined. Two tryptophan residues are located in the proximity of the C-terminal tail of Mad2 that is involved in binding to Cdc20. Thus, binding of Cdc20P1 may affect the tryptophan fluorescence of Mad2. The intensity of tryptophan fluorescence of Mad2 was indeed reduced by 70% upon the addition of Cdc20P1 to saturation. Based on the titration curve, the Kd of the Mad2-Cdc20P1 complex was measured to be 3.6±0.2 μM. This is consistent with the fact that binding of Cdc20P1 to Mad2 occurs with an intermediate exchange rate on the NMR time scale. The relative low affinity of Cdc20P1 toward Mad2 may also be the cause of the slow aggregation phenomenon of the Mad2-Cdc20P1 complex. Thus, Mad2 ligands with higher affinity for Mad2 are required for structural studies.

### Example 4

### Identification of Mad2-Binding Peptides (MBPs) Using Phage Display

Since Mad2 binds to a short peptide (Cdc20P1) within Cdc20, a phage display library containing 12-residue peptides with random sequences was screened.

Briefly, the library of phage, each displaying a different peptide sequence, was incubated with a polystyrene plate coated with purified Mad2 protein. The phage that did not bind to Mad2 was washed away by the TBST buffer (TBS+0.1% Tween-20). The Mad2-bound phage was eluted with 200 mM Glycine-HCl (pH 2.2). The eluted pool of phage was amplified and re-screened with Mad2. After this process was repeated four times, individual clones of phage were isolated and sequenced.

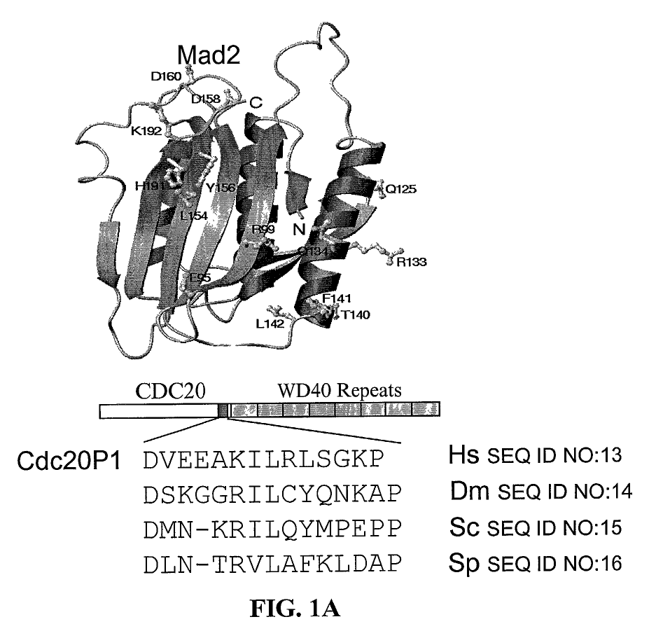

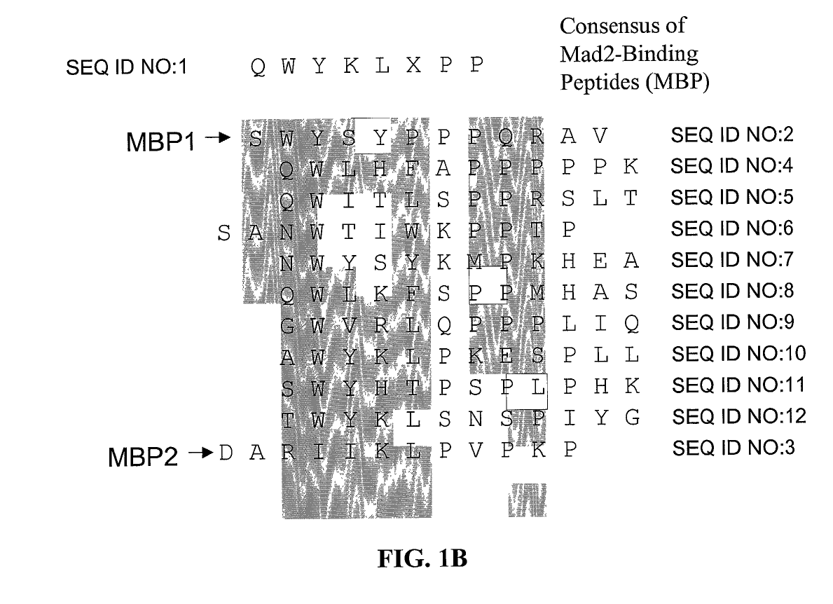

The amino acid sequences of the Mad2-binding peptides possess a consensus motif (FIG. 1A-B). The core of the motif consists of two hydrophobic residues, a basic residue, and a third hydrophobic residue. The hydrophobic residues tend to be aromatic amino acids. This core motif is generally followed by a proline-rich sequence. The sequences of the majority of peptides are divergent from that of Cdc20P1. However, one of the peptides, named MBP2, shares strong sequence similarity with Cdc20P1. A closer inspection reveals that Cdc20P1 and MBP2 contain a similar consensus as that found in the majority of Mad2-binding peptides, such as MBP1. The hydrophobic residues of the core motif in Cdc20P1 (highlighted in red) consist of leucines and isoleucines while residues at the same positions in MBP1 are aromatic residues. In fact, the sequence of the Mad2-binding domain of Cdc20 is not strictly conserved during evolution. As shown in FIG. 1A, the core Mad2-binding motifs of Fizzy (the Drosophila Cdc20), the S. cerevisiae Cdc20p, and Slp1 (the S. pombe Cdc20) contain an aromatic residue (Tyr or Phe) as the 3rd hydrophobic residue, similar to MBP1. There are also one or more prolines C-terminal to the core motif in all the Cdc20 sequences. Therefore, the Mad2-binding peptides identified through phage display contain similar core sequence elements as those found in Cdc20 proteins. It is very likely that MBP1 interacts with Mad2 in a manner similar to Cdc20P1.

### Example 5

### Structural Studies on the Mad2-MBP1 Complex

To confirm that MBP1 binds to Mad2, the inventors synthesized MBP1 and a control peptide (MBP1-rev) containing the reverse sequence of MBP1.

Addition of MBP1 caused dramatic chemical shift changes of the majority of signals in the ¹⁵N—¹³ H HSQC spectrum of Mad2. In contrast, the HSQC spectrum of Mad2 after the addition of MBP1-rev was identical with that of the free Mad2. These data indicate that MBP1 interacts specifically with Mad2.

To determine whether MBP1 and Cdc20 bind to the same site on Mad2. Mad2 was pre-incubated with an excess amount of Cdc20P1. MBP1 was then added to the pre-formed Mad2-Cdc20P1 complex. HSQC spectra were taken before and after the addition of MBP1. Prior to the addition of MBP1, Mad2 formed a complex with Cdc20P1 as indicated by the HSQC spectrum. After the addition of MBP1, the HSQC spectrum of the resulting sample looked identical to that of the Mad2-MBP1 complex. Therefore, MBP1 effectively displaced Cdc20P1 away from Mad2 and formed a complex with Mad2 in the presence of Cdc20P1. Thus, MBP1 and Cdc20P1 bind to a similar site on Mad2 and the affinity of MBP1 toward Mad2 is higher than that of Cdc20P1. However, it is also conceivable that MBP1 affects the binding of Cdc20P1 to Mad2 in an allosteric fashion.

Mad2-MBP1 complex was stable for extended periods of time and did not aggregate. Thus, the structure of the Mad2-MBP1 was determined by NMR. Briefly, a set of triple-resonance experiments, including HNCA, HN(CO)CA, HN(COCA)CB, and HN(CA)CB, were acquired on a sample of ¹⁵N/¹³C/²H-labeled Mad2 in complex with unlabeled MBP1 dissolved in H₂O. On the basis of these spectra, sequential assignments of more than 90% of the backbone resonances were obtained. To assign the ¹H and ¹³C resonances of side chains of Mad2, two 3D H(CC)(CO)NH and (H)C(C)(CO)NH spectra were acquired on a sample of 60% ²H and 100% ¹³C/¹⁵N-labeled Mad2 protein in complex with unlabeled MBP1. The analysis of these two spectra resulted in the assignment of about 50% of the side chain resonances. The sequential assignment of MBP1 was achieved through analysis of 3D ¹H/¹⁵N NOESY-HSQC and TOCSY-HSQC acquired on a sample of ¹⁵N-labeled MBP1 in complex with unlabeled Mad2. To obtain inter-molecular NOEs between Mad2 and MBP1, a 3D NOESY spectrum with ¹³C-editing in the dimension and ¹³C-filtering in the t3 (directly observed) dimension was acquired on a sample of ¹⁵N/¹³C-labeled Mad2 bound to unlabeled MBP1.

The sequential assignment of the backbone resonances of Mad2 allowed was used to identify NOEs involving backbone protons, such as HN-HN and HN-HA NOEs, through a partial analysis of the ¹⁵N-NOESY-HSQC spectrum of the ¹⁵N-labeled Mad2 bound to unlabeled MBP1. These NOEs, in combination of the intermolecular NOEs between Mad2 and MBP1, led to the determination of the secondary structure of the complex. Consistent with the dramatic chemical shift changes observed in the HSQC spectra, binding of MBP1 to Mad2 induces an extensive structural rearrangement. However, this conformational change mainly involves the N- and C-terminal regions of Mad2. The central region containing residues 20-160 maintains a similar structure. Therefore, a 3D model of the Mad2-MBP1 complex was calculated with a set of distance restraints containing the original restraints within the central region (residues 20-160) of the free Mad2, along with restraints derived from the newly identified intermolecular NOEs and the backbone NOEs that define the secondary structure of the complex.

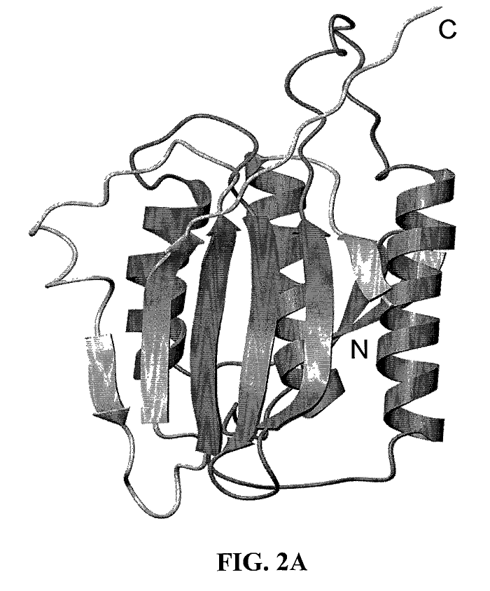

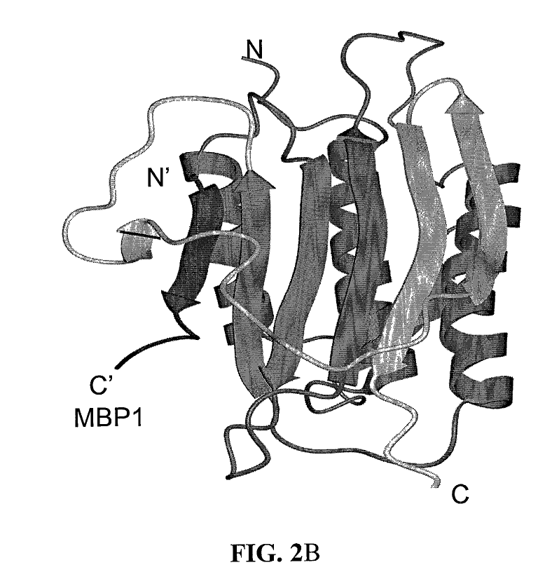

The overall architecture of the Mad2-MBP1 complex is similar to that of the free Mad2 (FIG. 2). It also consists of three layers: a central layer formed by three α-helices, a large β-sheet on one side, and a highly twisted β-hairpin on the other side. The major difference between the free and the peptide-bound forms of Mad2 lies in the arrangement of the large β-sheet. In the free Mad2 structure, the main P-sheet comprises six strands in a mixed parallel and anti-parallel configuration. In the structure of the Mad2-MBP1 complex, the main β-sheet consists of seven anti-parallel strands. This structural difference is caused by the incorporation of the MBP1 peptide as one of the strands (shown in red) and by the rearrangement of the C-terminal portion of Mad2, including the flexible C-terminal tail (shown in yellow in both structures for comparison). The last two strands and the C-terminal tail in the free Mad2 structure form three strands in the complex. A small strand adjacent to MBP1 lies at one edge of the new sheet while the other two strands form a β-hairpin that displaces the N-terminal strand in free Mad2 and define the other edge of the major sheet in the complex. Though the C-terminal region of Mad2 is required for binding to MBP1 and other peptides, this region does not directly contact the peptide in the complex. Instead, the conserved tail region, along with many other residues conserved during evolution, may be essential for the structural rearrangement triggered by peptide binding.

### Example 6

### Structure of Mad2 in Complex with a Cdc20-like Peptide

The structure of the Mad2-MBP2 complex was solved using the above methodology for Mad2-MBP1, with the exception that the stereospecific assignments of valine and leucine methyl groups are also obtained for the peptide from a high resolution ¹H—¹³C CT-HSQC spectrum acquired on a 10% ¹³C-labeled sample of MBP2 bound to Mad2.

Briefly, because MBP2 and Cdc20P1 have similar sequences, the structure of the Mad2-MBP2 complex is expected to resemble more closely to that of Mad2-Cdc20P1. In addition, the ¹H—¹⁵N HSQC spectra of ¹⁵N-Mad2-Cdc20P1 and ¹⁵N-Mad2-MBP2 were more similar to each other than to the ¹H—¹⁵N HSQC spectrum of ¹⁵N-Mad2-MBP1.

As expected, binding of MBP2 to Mad2 induced dramatic chemical shift changes of the majority of Mad2 signals in the HSQC spectrum. The HSQC spectrum of the Mad2-MBP2 complex closely resembled that of Mad2-Cdc20P1, suggesting that they adopt very similar structures. More importantly, the Mad2-MBP2 complex did not form high molecular weight aggregates after incubation at 30° C. for extended periods of time.

The relative affinity of Mad2-MBP2 was compared to Mad2-Cdc20P1 and Mad2-MBP1. When both MBP2 and Cdc20P1 were added to a sample of ¹⁵N-labeled Mad2, only the signals of Mad2-MBP2 were observed in the HSQC spectrum, indicating that MBP2 binds to Mad2 with a higher affinity than Cdc20P1. However, when both MBP1 and MBP2 were added to Mad2, the HSQC spectrum of the resulting sample contained two sets of signals of roughly equal intensity: one set of signals belonged to Mad2-MBP1 while the other set was from Mad2-MBP2. Therefore, MBP1 and MBP2 bind to Mad2 with very similar affinities, resulting in the formation of both complexes at equilibrium. Furthermore, the exchange between these two bound forms of Mad2 occurs with a rate that is slow on the NMR time scale.

### Example 7

### Kinetic Pathway of the Conformational Change of Mad2 Upon Peptide Binding

The structures of Mad2-MBP1 and Mad2-MBP2 reveal that binding of peptide ligands to Mad2 induce a dramatic conformational change of Mad2.

The hydrogen-deuterium exchange (HX) method has been widely used for the studies of protein folding (Englander et al., 2000 and Rumbley et al., 2001). The advantage of this method is the detection of folding intermediates that are infinitesimally populated at equilibrium. Though proteins exist predominantly in their folded state under native conditions, they constantly undergo global unfolding and refolding reactions. The global unfolding rate may be too slow to observe under native conditions. In contrast, the partial unfolding among defined secondary elements may occur with appreciable rates with the addition of low concentrations of denaturants or slightly elevated temperature. These local unfolding events are easily monitored by the so-called native-state HX method (Rumbley et al, 2001 and Fuentes et al., 1998).

Briefly, ¹⁵N-Mad2 are lyophilized and dissolved in D₂O to initiate the H/D exchange. A series of HSQC spectra each lasting 20 min is acquired over a period of 48 hrs. The H/D exchange rate of backbone amide protons is determined. Increasing concentrations of Guanidine-HCl (0-1 M, with 0.1 M increment) are added in the D₂O buffer and the amide H/D exchange rate is measured at each Guanidine-HCl concentration. The exchange rate of each amide proton is plotted against the concentration of denaturants. The protons residing in a particular folding unit (foldon) are expected to exhibit a similar profile with increasing concentrations of Guanidine-HCl. Therefore, by grouping the exchange rates of the amide protons, individual foldons of Mad2 are identified. These data reveal whether the N- and C-terminal regions in the free Mad2 structure indeed undergo rapid local unfolding (FIG. 2).

The same analysis is carried out on the Mad2-MBP1 and Mad2-MBP2 complexes. In this case, the interactions between β5 and β9 are expected to be the weakest link among the tertiary associations of secondary structure elements. If the backbone resonance assignment of Mad2-Cdc20P1 is accomplished, the Mad2-Cdc20P1 complex is also subjected to the HX analysis. Therefore, with HX experiments on the free Mad2 and the Mad2-peptide complexes, the kinetic pathway for the conformational change of Mad2 is defined. The intermediates observed in the kinetic pathway of the formation of Mad2-peptide complexes are related to the folding intermediates of Mad2. Therefore, these results indicate that recombinant Mad2 exists in both monomeric and oligomeric forms.

Protein residues essential for function are often conserved during evolution. It remains controversial whether the determinants of protein folding are also evolutionarily selected. In the case of Mad2, its inherent conformational flexibility might be conserved both for its function (binding to Cdc20) and for its proper folding. In fact, many of the surface conserved residues of Mad2 proteins from different organisms reside in strands β5, β6, new β7, β8, and β9, suggesting that the mechanism of the conformational switch might be conserved.

### Example 8

### Mutagenesis Analysis of Mad2 and MBP2

To illustrate that the C-terminal portion (residues 158-205) of Mad2 is a relatively independent folding unit, two Mad2 deletion mutants are constructed. One mutant spans residues 1-157 while the other contains residues 158-205. Both of these two fragments are expressed and purified individually, and their structures are analyzed by NMR. The ¹H—¹⁵N HSQC spectra of the two fragments reveals whether one or both retain a defined tertiary fold. If so, the structures of the two fragments are determined by NMR. In addition, the thermostability of both the intact Mad2 monomer and the Mad21-157 fragment is analyzed using circular dichroism (CD) spectroscopy. If the Mad21-157 mutant retains a similar stable fold as that of the intact protein, it indicates that the C-terminal portion is dispensable for the folding and stability of Mad2. This, along with the HX data, indicate the proposed kinetic pathway of the Mad2 structural rearrangement.

Peptide ligands of Mad2 participate in the formation of P sheet in the complex. To examine the relative contributions of the backbone and the side chains of MBP2 to the binding affinity, alanine scanning mutagenesis of MBP2 is performed. A total of 12 mutant peptides with each position of MBP2 changed to alanine will be synthesized. Because MBP2 does not contain tryptophans, the affinities of these peptides toward Mad2 are determined by tryptophan fluorescence perturbation experiments. The energetic contribution of each side chain is calculated. Similar analysis are performed for MBP1. However, the affinity between Mad2 and MBP1 is measured using a different analysis because of the presence of tryptophans in MBP1.

### Example 9

### Identification of Ligands with Higher Affinity Toward Mad2

Phage display library with Mad2 was screened and several clones were sequenced and analyzed using ELISA-type assays.

The phage display library used in the screen contains 2×10⁹ individual clones. However, there are 20¹² (4.1×10¹⁵) possible combinations of amino acid sequences for a 12-residue peptide. Therefore, the library represents only a very minor portion of the sequence space of 12 amino acid peptides. To overcome this problem, a biased random phage display peptide library was construced by cloning a library of oligonucleotides into the M13KE gIII vector (New England Biolabs) per instruction of the manufacturer. The library encodes peptides of the following pattern: XWYKLXXPXXXX (SEQ ID NO:17), where X indicates any residue. The WYKLXXP (SEQ ID NO:18) motif was found in several Mad2-binding ligands. By making this motif invariable in all the peptides, the inventors reduced the positions within the peptides that are randomized to 7, thereby limiting the possible combination of sequences to 20⁷ (1.3×10⁹). Therefore, the new biased library only needs 2×10⁹ individual clones to completely sample the sequence space. These peptides are expressed as extreme N-terminal fusions with the coat protein (pIII) of the bacteriophage, and are thus displayed on the surface of the phage. The purified recombinant Mad2 protein is used as bait to screen the phage display library as described above. Screening of this biased library may produce even higher affinity peptide ligands for Mad2.

By coincidence, the MBP1 sequence was isolated again and the MBP1 phage clone gave an ELISA reading of 0.62 (OD at 410 nm). Several clones in this new group displayed higher ELISA OD readings than MBP1, indicating that they bind with higher affinity to Mad2. For example, clone 11 (GWWHIPSPVLRP; SEQ ID NO:19) had an OD reading of 1.37. Because the binding affinity did not bear a linear relationship with the ELISA value, clone 11 is expected to bind to Mad2 with much higher affinity. The peptides with higher ELISA readings than MBP1 are synthesized. As these peptides, including MBP1, contain tryptophan in their sequences, tryptophan fluorescence assay cannot be used to measure the affinities of these peptides toward Mad2. Alternative assays are developed and used.

The peptide ligands are synthesized with the following additional sequences at the C-termini: GGGC. The cysteines are used for coupling the peptides to the sensor chip of BIAcore with the thiol coupling approach (Malmqvist et al., 1999; Fivash et al., 1998; O'Shannessy et al., 1994). The preliminary structure of Mad2-MBP1 indicates that coupling of the C-termini of the ligands onto a solid support is unlikely to interfere with their binding to Mad2. Once the coupling of the ligands to the chip is accomplished, purified Mad2 protein is injected in solution over the chip under continuous flow conditions. Binding of Mad2 to the immobilized ligands changes the refractive index of the chip, resulting in a surface plasmon resonance signal that is used to extrapolate the on- and off-rates of the binding. The affinity between Mad2 and Cdc20P1 is measured with this method and to compare the result from BIAcore with the affinity determined by the fluorescence data.

The Mad2 ligands form an extended P-strand conformation when bound to Mad2. It is contemplated that the peptide backbones of these ligands contribute significantly to the binding affinities toward Mad2. Therefore, to obtain non-peptide organic compounds by screening Mad2 against compound libraries created by combinatorial synthesis, one approach is to preserve the abilities of these compounds to form hydrogen bonds with the neighboring strands in Mad2. The polypyrrolinone scaffold developed by Amos Smith and coworkers is a good starting point (Smith et al., 2000; Simithe et al., 1996). These compounds are capable of adopting the P-strand conformation. Also, it was demonstrated that compounds with this scaffold can be synthesized on solid support, making it feasible to synthesize combinatorial libraries with various side chains (Smith et al., 2000).

### Example 10

### Inhibition of Mad2 by MBP1 and other Mad2-Binding Peptides

Mad2 is required for the proper function of the mitotic checkpoint. Disruption of the Mad2 gene in mice by homologous recombination causes embryonic lethality (Dobles et al., 2000). The Mad2-null cells show a gross chromosome mis-segregation phenotype, resulting in apoptosis (Dobles et al., 2000). Similar effects have been observed in Drosophila for other checkpoint genes, such as Bub1 (Basu et al., 1999). These results suggest that inactivation of the mitotic checkpoint signaling leads to cell death due to improper chromosome segregation. Therefore, a complete inhibition of Mad2 function in cancer cells by chemical agents is predicted to effectively promote apoptosis.

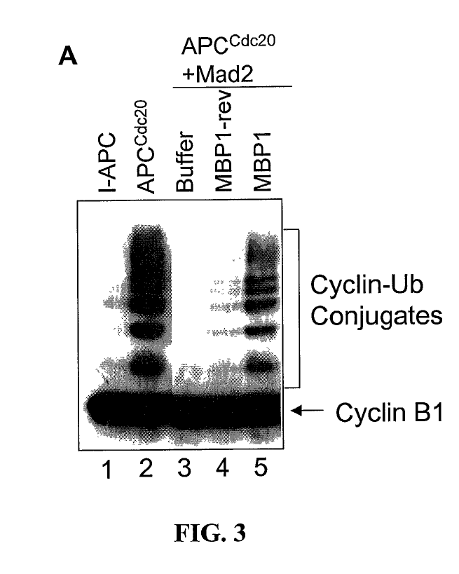

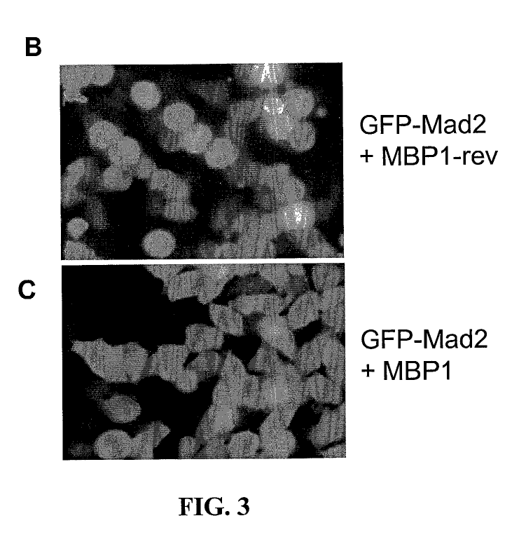

The inventors showed that MBP1 binds to Mad2 with high affinity and binding of MBP 1 prevents the interaction between Mad2 and the Cdc20P1 peptide. To determine whether binding of MBP1 affects the association of Mad2 with the intact Cdc20, the inventors performed an in vitro APC ubiquitination assay. In this assay, a Myc-epitope tagged N-terminal fragment of human cyclin B1 was used as the substrate. Purified recombinant human E1, UbcH10 (E2), ATP, and ubiquitin were also included (FIG. 3A). In the presence of active APC (E3), ubiquitin was ligated to the lysine side chains of cyclin B1, forming cyclin-ubiquitin conjugates with isopeptide linkages. The reaction mixture was then analyzed by SDS-PAGE followed by immuno-blotting with an anti-Myc antibody. The appearance and the intensity of the higher molecular weight bands were then used as the criteria for the activity of APC.

As shown in FIG. 3B, APC purified from interphase Xenopus egg extracts possessed only basal level activity. Addition of the APC activator, Cdc20, greatly stimulated the activity of APC. A pre-incubation of Cdc20 with Mad2 effectively inhibited the ubiquitination activity of APC. Interestingly, MBP1 at 100 μM prevented Mad2 from inhibiting APCCdc20 while the control peptide, MBP1-rev, did not have any effect (FIG. 3C). Therefore, MBP1 can block the biochemical function of Mad2 in vitro.

### Example 11

### MBP1 Reverses the Mitotic Arrest Phenotype of Mad2 Overexpression in HeLa Cells

Overexpression of Mad2 in HeLa cells causes a prolonged mitotic arrest because Mad2 at elevated levels inhibits APCCdc20 in these cells in the absence of spindle damage. As shown in FIG. 3B-C, co-transfection of a plasmid encoding GFP-Mad2, together with a plasmid encoding the MBP1-rev peptide, led to accumulation of cells in mitosis, as revealed by cell shape. The cells round up when they undergo mitosis while the interphase cells are flat. In contrast, cells transfected with the GFP-Mad2 vector and a plasmid encoding MBP1 did not arrest in mitosis, indicating that MBP1 blocked the function of Mad2 in living cells. The cell cycle status of the transfected cells was also confirmed by DNA-staining and by FACS analysis.

### Example 12

### Cellular Effects of the Mad2 Inhibitors

Inhibitors of Mad2 may be used as potent anti-cancer drugs. The effects of the Mad2-binding peptides are examined on mammalian tissue culture cells. Two different approaches are used to deliver the peptides into cells.

First, duplex oligonucleotides encoding these peptides are cloned into a mammalian expression vector with a strong promoter, such as the CMV promoter. The plasmids are introduced into cells through standard transfection techniques. Expression of these peptides either alone or as fusion proteins with GFP driven by the strong promoter should result in the accumulation of the peptides to relatively high concentrations (100 nM-1 μM) inside the cell.

In a second method, peptides are synthesized that consist of a small segment of the HIV-TAT protein at the N-termini, a flexible poly-glycine linker, and the Mad2-binding sequences at the C-terminal region. Fusion of the HIV-TAT peptide (YGRKKRRQRRR; SEQ ID NO:20) or related peptides to the N-termini of proteins leads to efficient delivery of the resulting fusion proteins to the interior of mammalian cells (Wender et al., 2000). A fusion peptide between TAT and one of Mad2 ligands is made and then labeled with a fluorescent tag. The accumulation of this peptide in cells is monitored using a fluorescent microscope. The intensity of the fluorescence is used to estimate the cellular concentration of the peptide.

In both approaches, peptides that contain the reverse amino acid sequences of the Mad2 ligands are introduced into cells and used as controls. The phenotype of the peptide-expressing cells is examined by directly observing them under an inverted fluorescent microscope. The morphology of DNA of live cells is visualized by staining the cells with Hoechst 33342. This reveals any potential mitotic or apoptotic phenotype. To determine their cell cycle status more quantitatively, the cells are fixed with ethanol, stained with propidium iodide, and subjected to FACS analysis.

### Example 13

### Combined Therapies

Mad2 inhibitory peptides is combined with with a microtubule poisons, such as nocodazole and Taxol. Taxol is a new chemotherapeutic agent for the treatment of advanced breast cancer, ovarian cancer, and lung cancer (Crown et al., 2000). It also shows promising results in clinical trials for the treatment of other forms of cancer (Crown et al., 2000). The molecular mechanism of Taxol as an anti-cancer drug is well-understood. A plethora of evidence indicates that the cytotoxic effect of Taxol is mainly due to its ability to induce apoptosis in cancer cells (Wang et al., 2000). Through direct binding to tubulin, Taxol stabilizes the microtubule fibers of the mitotic spindle (Amos et al., 1999). Many cancer cells have defective mitotic checkpoint. Therefore, these cells will attempt to divide in the absence of a functional spindle due to Taxol treatment, leading to cell death. Certain tumor cells are resistant to Taxol, which may be due to the presence of an intact mitotic checkpoint in these cells (Crown et al., 2000). Exposure of cancer cells with an intact mitotic checkpoint to Taxol will trigger this checkpoint and cause a prolonged mitotic arrest. Upon the removal of Taxol, these cells may recover and continue to divide. Thus, cancer cells with proper mitotic checkpoint function may be more resistant to Taxol. It is envisioned that a disruption of the mitotic checkpoint in cancer cells may prime them for killing by Taxol.

Mad2 inhibitors are added to HeLa cells, in combination with various concentrations of Taxol. The survival rate of the cells are measured.

All of the COMPOSITIONS and/or METHODS and claimed herein can be made and executed without undue experimentation in light of the present disclosure. While the compositions and methods of this invention have been described in terms of preferred embodiments, it will be apparent to those of skill in the art that variations may be applied to the COMPOSITIONS and/or METHODS and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.

EPO 0273085

U.S. Pat. No. 4,418,068

U.S. Pat. No. 4,870,287

U.S. Pat. No. 4,664,911

U.S. Pat. No. 4,792,447

U.S. Pat. No. 5,712,097

U.S. Pat. No. 5,045,451

U.S. Pat. No. 5,220,007

U.S. Pat. No. 5,284,760

U.S. Pat. No. 5,279,721

U.S. Pat. No. 5,354,671

U.S. Pat. No. 5,366,878

U.S. Pat. No. 5,635,377

U.S. Pat. No. 5,578,706

U.S. Pat. No. 5,767,072

U.S. Pat. No. 5,635,377

U.S. Pat. No. 5,760,395

U.S. Pat. No. 5,789,166

U.S. Pat. No. 5,798,208

WO 84/03564

WO 88/10315

WO 89/06700

WO 90/07641

WO 01/00825

Abrieu, A., et al., Cell 102, 817-826, 2000.

Amos, L. A. et al., Chem Biol 6, R65-69, 1999.

Arcone, et al., Nucl. Acids Res., 16(8): 3195-3207, 1988.

Baichwal and Sugden, In: Gene Transfer, pp. 117-148, 1986.

Bakke et al., Cell 63:727-716, 1990.

Barany and Merrifield, The Peptides, pp. 1-284, 1979.

Bardet, G. Thesis 479, (1920).

Bartlett et al., Proc. Nat'l Acad. Sci. USA, 93:8852-8857, 1996.

Basu, J., et al., J Cell Biol 146, 13-28, 1999.

Bax, A. et al., Acc Chem Res 26, 131-138, 1993.

Benvenisty and Neshif, Proc. Nat'l Acad. Sci. USA, 83:9551-9555, 1986.

Biedl, A. Dtsch. Med. Wschr. 48, 1630, 1922.

Brunger, A. T., et al., Acta Crystallogr D Biol Crystallogr 54, 905-921, 1998.

Burke, D. J. Curr Opin Genet Dev 10, 26-31, 2000.

Cahill, D. P., et al., Nature 392, 300-303 (1998).

Capaldi et al., Biochem. Biophys. Res. Comm., 76:425, 1977

Carter and Flotte, Ann. N.Y. Acad. Sci., 770:79-90, 1995.

Chan, G. K., et al., J Cell Biol 143, 49-63, 1998.

Chan, G. K., et al., J Cell Biol 146, 941-954, 1999.

Chatterjee, et al., Ann. N.Y. Acad. Sci., 770:79-90, 1995.

Chen and Okayama, Mol. Cell Biol., 7:2745-2752, 1987.

Chen, R. H., et al., J Cell Biol 143, 283-295, 1998.

Chen, R. H., et al., Mol Biol Cell 10, 2607-2618, 1999.

Chen, R. H., et al., Science 274, 242-246, 1996.

Ciosk, R., et al., Cell 93, 1067-1076, 1998.

Clarke, D. J. et al., Bioessays 22, 351-363, 2000.

Clore, G. M. et al., Meth Enzymol 239, 349-363, 1994.

Clore, G. M. et al., Nat Struct Biol 4 Suppl, 849-853, 1997.

Clore, G. M. et al., Curr Opin Chem Biol 2, 564-570, 1998.

Clore, G. M. et al., Trends Biotechnol 16, 22-34, 1998.

Coffin, In: Virology, ed., New York: Raven Press, pp. 1437-1500, 1990.

Cornilescu, G., et al., J Biomol NMR 13, 289-302, 1999.

Coupar et al., Gene, 68:1-10, 1988.

Crown, J. et al., Lancet 355, 1176-1178, 2000.

Czisch, M. et al., J Magn Reson 134, 158-160, 1998.

Delaglio, F., et al., J Biomol NMR 6, 277-293, 1995.

Dobles, M., et al., Cell 101, 635-645, 2000.

Englander, S. W. Annu Rev Biophys Biomol Struct 29, 213-238, 2000.

Fang, G., et al., Genes Dev 12, 1871-1883, 1998.

Fang, G., et al., Mol Cell 2, 163-171, 1998.

Ferkol et al., FASEB J., 7:1081-1091, 1993.

Ferrari et al., J. Virol., 70:3227-3234, 1996.

Fisher et al., J. Virol., 70:520-532, 1996.

Fivash, M., et al., Curr Opin Biotechnol 9, 97-101, 1998.

Flotte et al., Proc. Nat'l Acad. Sci. USA, 90:10613-10617, 1993.

Fuentes, E. J. et al., Biochemistry 37, 3687-3698, 1998.

Gardner, K. H. et al., Annu Rev Biophys Biomol Struct 27, 357-406, 1998.

Gardner, R. D. et al., Trends Cell Biol 10, 154-158, 2000.

Ghosh and Bachhawat, In: Liver Diseases, Targeted Diagnosis and Therapy Using Specific Receptors and Ligands.

Goodman et al., Blood, 84:1492-1500, 1994.

Gopal, Mol. Cell Biol., 5:1188-1190, 1985.

Graham and van der Eb, Virology, 52:456-467, 1973.

Hageforden et al., J. Biol. Chem. 266:6015, 1991.

Hardwick, K. G., et al., J Cell Biol 148, 871-882, 2000.

Hardwick, K. G., et al., Science 273, 953-956, 1996.

Harland and Weintraub, J. Cell Biol., 101:1094-1099, 1985.

Harlow et al., Mol Cell Biol. 5:1605, 1985.

Hay et al., J. Mol. Biol., 175:493-510, 1984.

Hearing and Shenk, J. Mol. Biol. 167:809-822, 1983.

Hearing et al., J. Virol., 67:2555-2558, 1987.

Howell, B. J., et al., J Cell Biol 150, 1233-1250, 2000.

Hoyt, M. A., et al., Cell 66, 507-517, 1991.

Hwang, L. H., et al., Science 279, 1041-1044, 1998.

Jablonski, S. A., et al., Chromosoma 107, 386-396, 1998.

Jin, D. -Y., et al., Cell 93, 81-91, 1998.

Johnson et al., Peptide Turn Mimetics” IN: Biotechnology And Pharmacy, 1993.

Johnson, B. A. et al., J Biomol NMR 4, 603-614, 1994.

Joki et al., Human Gene Ther., 6:1507-1513, 1995.

Kageyama et al., J Biol. Chem., 262(5):2345-2351, 1987.

Kaneda et al., Science, 243:375-378, 1989.

Kaplitt et al., Nat'l Genet., 8:148-153, 1994.

Kato et al, J. Biol. Chem., 266:3361-3364, 1991.

Kay, L. E. Biochem Cell Biol 75, 1-15, 1997.

Kay, L. E. et al., Curr Opin Struct Biol 7, 722-731, 1997.

Kessler et al., Proc. Nat'l Acad. Sci. USA, 93:14082-14087, 1996.

Kim, S. H., et al., Science 279, 1045-1047, 1998.

King, R. W., et al., Cell 81, 279-288, 1995.

Klein et al., Nature, 327:70-73, 1987.

Koeberl et al., Proc. Nat'l Acad. Sci. USA, 94:1426-1431, 1997.

Kubota et al., Biochem. and Biophy. Res. Comm. 162:963, 1989.

Lanford et al., Cell 46:575, 1986.

Lee, H., et al., Mol Cell 4, 1-10, 1999.

Letoumeur et al., Cell 69:1183, 1990.

Levrero et al., Gene, 101:195-202, 1991.

Li, R. et al., Cell 66, 519-531, 1991.

Li, Y. et al., Science 274, 246-248, 1996.

Li, Y., et al., Proc Natl Acad Sci USA 94, 12431-12436, 1997.

Lin, Y. et al., J Biomol NMR 15, 227-239, 1999.

Losada, A., et al., Genes Dev 12, 1986-1997, 1998.

Losada, A., et al., J Cell Biol 150, 405-416, 2000.

Luo, X., et al., Nat Struct Biol 7, 224-229, 2000.

Malmqvist, M. Biochem Soc Trans 27, 335-340, 1999.

Mann et al., Cell, 33:153-159, 1983.

Martinez-Exposito, M. J., et al., Proc Natl Acad Sci USA 96, 8493-8498, 1999.

McCown et al., Brain Res., 713:99-107, 1996.

Merrifield, Science, 232: 341-347, 1986.

Michaelis, C., et al., Cell 91, 35-45, 1997.

Mizukami et al, Virology, 217:124-130, 1996.

Munro et al., Cell 48:899-907, 1987.

Nasmyth, K. Trends Biochem Sci 24, 98-104, 1999.

Nasmyth, K., et al., Science 288, 1379-1385, 2000.

Nicolas and Rubenstein, In: Vectors: A survey of molecular cloning vectors and their uses, pp. 493-513, 1988.

Nicolau and Sene, Biochim. Biophys. Acta, 721:185-190, 1982.

Nicolau et al., Methods Enzymol., 149:157-176, 1987.

Nietlispach, D., et al., J Am Chem Soc 118, 407-415, 1996.

Nilges, M., et al., J Mol Biol 269, 408-422, 1997.

Olivierio et al., EMBO J., 6(7):1905-1912, 1987.

O'Shannessy, D. J. Curr Opin Biotechnol 5, 65-71, 1994.

Perales et al., Proc. Nat'l Acad. Sci. 91:4086-4090, 1994.

Pervushin, K., et al., Proc Natl Acad Sci USA 94, 12366-12371, 1997.

Ping et al., Microcirculation, 3:225-228, 1996.

Poli and Cortese, Proc. Nat'l Acad. Sci. USA, 86:8202-8206, 1989.

Potter et al., Proc. Nat'l Acad. Sci. USA, 81:7161-7165, 1984.

Prowse and Baumann, Mol Cell Biol, 8(1):42-51, 1988.

Pugsley, A. P., Protein Targeting, Academic Press, Inc. 1989.

Radler et al., Science, 275:810-814, 1997

Ridgeway, In: Vectors: A survey of molecular cloning vectors and their uses, pp. 467-492, 1988.

Rieder, C. L., et al., J Cell Biol 130, 941-948, 1995.

Rippe et al., Mol. Cell Biol., 10:689-695, 1990.

Roberts, et al., Mol Cell Biol 14, 8282-8291, 1994.

Ron, et al., Mol. Cell. Biol., 2887-2895, 1991.

Roux et al., Proc. Nat'l Acad. Sci. USA, 86:9079-9083, 1989.

Rumbley, J., et al., Proc Natl Acad Sci USA 98, 105-112, 2001.

Salzmann, M., et al., Proc Natl Acad Sci USA 95, 13585-13590, 1998.

Sambrook et al., In: Molecular Cloning: A Laboratory Manual, 1989.

Samulski et al., J. Virol., 61(10):3096-3101, 1987.

Sattler, M., et al., Prog NMR Spectr 34, 93-158, 1999.

Seomi et al., J. Virology 64:1803, 1990.

Shah, J. V. et al., Cell 103, 997-1000, 2000.

Siomi et al., Cell 55:197, 1988.

Smith, A. B., et al., Bioorg Med Chem 4, 1021-1034, 1996.

Smith, A. B., et al., Org Lett 2, 2037-2040, 2000.

Stanton, L. W. et al., Proc. Natl. Acad. Sci U.S.A. 83:1772, 1986.

Stein, E. G., et al., J Magn Reson 124, 154-164, 1997.

Stewart and Young, Solid Phase Peptide Synthesis, 2d. ed., Pierce Chemical Co., 1984.

Takagi et al., Gastroenterology, 111: 1369, 1996.

Tam et al., J. Am. Chem. Soc., 105:6442, 1983.

Tanaka, T., et al., Nat Cell Biol 2, 492-499, 2000.

Tang et al., J. Biol. Chem. 207: 10122

Taylor, S. S. et al., Cell 89, 727-735, 1997.

Taylor, S. S., et al., J Cell Biol 142, 1-11, 1998.

Temin, In: Gene Transfer, Kucherlapati (ed.), New York: Plenum Press, pp. 149-188, 1986.

Tibbetts Cell, 12:243-249, 1977.

Tur-Kaspa et al., Mol. Cell Biol., 6:716-718, 1986.

Wagner et al., Proc. Nat'l Acad. Sci. 87, 9:3410-3414, 1990.

Walther and Stein, J. Mol. Med., 74:379-392, 1996.

Wang, T. H., et al., Cancer 88, 2619-2628, 2000.

Watt et al., Proc. Nat'l Acad. Sci., 83(2): 3166-3170, 1986.

Wender, P. A., et al., Proc Natl Acad Sci USA 97, 13003-13008, 2000.

Wilson et al., Mol. Cell. Biol., 6181-6191, 1990.

Wong et al., Gene, 10: 87-94, 1980.

Wu and Wu, Adv. Drug Delivery Rev., 12:159-167, 1993.

Wu and Wu, J. Biol. Chem., 262:4429-4432, 1987.

Wu and Wu, Biochem., 27:887-892, 1988.

Wu et al., Genomics, 4:560, 1989.

Wu et al., pp. 87-104, 1991.

Xiao et al., J. Virol., 70:8098-8108, 1996.

Yang et al., Proc. Nat'l Acad. Sci. USA, 87:9568-9572, 1990.

Yao, X., et al., Nat Cell Biol 2, 484-491, 2000.

Yu, H., et al, Science 279, 1219-1223, 1998.

Zechner et al., Mol. Cell. Biol., 2394-2401, 1988.

What is claimed is:

1. A method of inhibiting Mad2 function comprising contacting a Mad2 protein with a peptide that binds Mad2.

2. The method of claim 1, wherein said peptide is 9 to about 20 residues in length.

3. The method of claim 2, wherein said peptide is 12 residues in length.

4. The method of claim 2, wherein said peptide comprises a core sequence represented by the formula X₁X₂X₃X₄X₅X₆X₇X₈X₉, wherein:
X₁ can be any amino acid;
X₂ and X₃ are hydrophobic residues;
X₄ is a basic residue;
X₅ is a hydrophobic residue; and
at least one of X₆ to X₉ is P.

5. The method of claim 4, wherein at least two of X₆ to X₉ are P.

6. The method of claim 4, wherein said peptide comprise at least on other P.

7. The method of claim 1, wherein the peptide comprises the sequence QWYKLX₆PP, SWYSYPPPQRAV, or DARIIKLPVPKP.

8. The method of claim 1, wherein said peptide is present in a molar excess of Mad2.

9. The method of claim 1, wherein said peptide is present in a 5-fold molar excess of Mad2.

10. The method of claim 1, wherein said peptide is present in a 10-fold molar excess of Mad2.

11. The method of claim 1, wherein said peptide is present in a 100-fold molar excess of Mad2.

12. The method of claim 1, wherein said peptide is delivered to a cell comprising said Mad2.

13. The method of claim 12, wherein said peptide is encapsulated in a liposome.

14. The method of claim 1, wherein a nucleic acid encoding said peptide and a promoter is delivered to a cell comprising said Mad2.

15. The method of claim 14, wherein said promoter is selected from the group consisting of CMV IE, RSV, and SV40 large T.

16. The method of claim 14, wherein said nucleic acid further comprises a polyadenylation signal.

17. The method of claim 14, wherein said nucleic acid is located in a viral vector.

18. The method of claim 17, wherein said viral vector is selected from the group consisting of retrovirus, adenovirus, adeno-associated virus, vaccinia virus, herpesvirus and polyoma virus.

19. The method of claim 1, wherein said Mad2 is located in a cancer cell.

20. The method of claim 19, further comprising contacting said cell with a DNA damaging agent.

21. The method of claim 20, wherein said DNA damaging agent is radiation.

22. The method of claim 21, wherein said radiation is x-irradiation, γ-irradiation, uv-irradiation, and microwave irradiation.

23. The method of claim 20, wherein said DNA damaging agent is a DNA damaging chemotherapeutic agent.

24. The method of claim 23, wherein said chemotherapeutic agent is a microtubule inhibitor or an anti-mitotic agent.

25. The method of claim 19, further comprising contacting said cancer cell with taxol.

26. The method of claim 1, wherein said peptide is linked to a nuclear targeting molecule.

27. The method of claim 26, wherein said nuclear targeting molecule is an SV40 nuclear localization signal.

28. A method of inhibiting Mad2 function comprising contacting a Mad2 protein with a peptide-mimic that binds to Mad2.

29. A method of inhibiting cancer cell proliferation comprising contacting a Mad2 protein with a peptide or peptide-mimic that binds to Mad2.

30. The method of claim 29, wherein said cancer cell is killed.

31. The method of claim 29, wherein said cancer cell is a prostate cancer cell, a breast cancer cell, a lung cancer cell, a brain cancer cell, a liver cancer cell, a pancreatic cancer cell, a stomach cancer cell, a colon cancer cell, an ovarian cancer cell, a testicular cancer cell, a head & neck cancer cell, a throat cancer cell and an esophageal cancer cell.

32. A method of treating cancer in a subject comprising administering to cancer cells of said subject a peptide or peptide-mimic that binds to Mad2.

33. The method of claim 31, wherein said subject is a human.

34. The method of claim 32, further comprising administering to said patient a second cancer therapy.

35. The method of claim 34, wherein said second cancer therapy is a DNA damaging agent.

36. The method of claim 35, wherein said DNA damaging agent is ionizing radiation.

37. The method of claim 35, wherein said DNA damaging agent is a chemotherapeutic agent.

38. The method of claim 34, wherein said second cancer therapy is taxol.

39. A method of screening for an anti-cancer agent comprising:
(a) providing a target polypeptide comprising at least the cdc20 binding domain of Mad2;
(b) contacting said target polypeptide with a candidate substance;
(c) determining the binding of said candidate substance to said target polypeptide; and
(d) in case of positive target polypeptide binding, screening for an anti-cancer effect.

40. The method of claim 39, wherein said candidate substance is a peptide.

41. The method of claim 40, wherein said peptide is selected from a peptide library.

42. The method of claim 39, wherein step (d) comprises admixing said candidate substance with a cancer cell and measuring one or more characteristics of said cancer cell.

43. The method of claim 42, wherein said characteristics include cell growth, cell viability, cell shape or cell differentiation.

44. The method claim 40, wherein step (d) comprises contacting an expression vector encoding said peptide with a cancer cell and measuring one or more characteristics of said cancer cell.

45. The method of claim 39, wherein said target peptide is expressed on the surface of a phage.


SEQUENCE LISTING (20 sequences)

SEQ ID NO: 1 - protein, 8 aa, Artificial Sequence
  Synthetic Peptide
QWYKLXPP

SEQ ID NO: 2 - protein, 12 aa, Artificial Sequence
  misc_feature (1)..(12): synthetic peptide
SWYSYPPPQRAV

SEQ ID NO: 3 - protein, 12 aa, Artificial Sequence
  misc_feature (1)..(12): synthetic peptide
DARIIKLPVPKP

SEQ ID NO: 4 - protein, 12 aa, Artificial Sequence
  misc_feature (1)..(12): synthetic peptide
QWLHFAPPPPPK

SEQ ID NO: 5 - protein, 12 aa, Artificial Sequence
  misc_feature (1)..(12): synthetic peptide
QWITLSPPRSLT

SEQ ID NO: 6 - protein, 12 aa, Artificial Sequence
  misc_feature (1)..(12): synthetic peptide
SANWTIWKPPTP

SEQ ID NO: 7 - protein, 12 aa, Artificial Sequence
  misc_feature (1)..(12): synthetic peptide
NWYSYKMPKHEA

SEQ ID NO: 8 - protein, 12 aa, Artificial Sequence
  misc_feature (1)..(12): synthetic peptide
QWLKFSPPMHAS

SEQ ID NO: 9 - protein, 12 aa, Artificial Sequence
  misc_feature (1)..(12): synthetic peptide
GWVRLQPPPLIQ

SEQ ID NO: 10 - protein, 12 aa, Artificial Sequence
  misc_feature (1)..(12): synthetic peptide
AWYKLPKESPLL

SEQ ID NO: 11 - protein, 12 aa, Artificial Sequence
  misc_feature (1)..(12): synthetic peptide
SWYHTPSPLPHK

SEQ ID NO: 12 - protein, 12 aa, Artificial Sequence
  misc_feature (1)..(12): synthetic peptide
TWYKLSNSPIYG

SEQ ID NO: 13 - protein, 14 aa, Homo sapiens
DVEEAKILRLSGKP

SEQ ID NO: 14 - protein, 15 aa, Drosophila
DSKGGRILCYQNKAP

SEQ ID NO: 15 - protein, 14 aa, S. Cerevisiae
DMNKRILQYMPEPP

SEQ ID NO: 16 - protein, 14 aa, S. Pombe
DLNTRVLAFKLDAP

SEQ ID NO: 17 - protein, 12 aa, Artificial Sequence
  misc_feature (1)..(1): X is any
XWYKLXXPXXXX

SEQ ID NO: 18 - protein, 7 aa, Artificial Sequence
  misc_feature (4)..(5): X is any
WYKLXXP

SEQ ID NO: 19 - protein, 12 aa, Artificial Sequence
  misc_feature (1)..(12): Synthetic Peptide
GWWHIPSPVLRP

SEQ ID NO: 20 - protein, 11 aa, HIV-TAT PROTEIN
YGRKKRRQRRR